(19) United States
(12) Patent Application Publication
(10) Pub. No.: US 2026/0179765 A1
(43) Pub. Date: Jun. 25, 2026

(54) SYSTEMS, DEVICES AND METHODS FOR MANAGING MEDICAL APPLICATIONS AND MEDICAL COMPUTER ASSIGNMENTS USING AN EXTERNAL COMPUTER

(71) Applicant: 3D BRIDGE SOLUTIONS INC., Vancouver (CA)

(72) Inventors: Gary MOUSSEAU, Waterloo (CA); Karima BAWA, Vancouver (CA)

(21) Appl. No.: 19/317,553

(22) Filed: Sep. 3, 2025

Related U.S. Application Data
(60) Provisional application No. 63/736,449, filed on Dec. 19, 2024.

(51) Int. Cl. G16H 40/40; G16H 10/65; G16H 30/20

(52) U.S. Cl. CPC G16H 40/40; G16H 10/65; G16H 30/20

(57) ABSTRACT

Embodiments relate to systems and methods for assigning a medical tracking application to a client device to provide codified medical instructions and receive medical activity about the client device. An external computer manages client device assignments that execute the medical tracking applications. Embodiments relate to systems and methods for improved security and activity tracking. For example, an external computer may be physically separate from the client's device, allowing for a separate control point to add security and detectability elements to medical events that are taking place.

### CROSS-REFERENCE TO RELATED APPLICATION

This application claims priority to and the benefit of U.S. Provisional Patent Application No. 63/736,449 filed Dec. 19, 2024, the entire contents of which is hereby incorporated by reference.

### FIELD

Embodiments relate to systems and methods for using an external computer to assign a medical tracking application to a client to provide codified medical instructions and receive medical activity about the client. The external computer also manages medical computer assignments that execute the medical tracking applications. The external computer being physically separate from the medical computer allows for a separate control point to add security and detectability elements to medical events that are taking place.

### INTRODUCTION

The use of medical tracking applications is widespread and there is a growing use for personal care. Professionals and caregivers who try to use these medical tracking applications for clients and loved ones may encounter the problem of lacking control over usage parameters and real-time guidance on usage of the medical tracking applications. These applications offer direct configurations that are typically directly set up by the client or caregiver on the computer hosting the applications with provided instructions. As a result, the process of setting up the application may be erroneous or overly complicated. Mistakes relating to medication consumption times can lead to dangerous results. Another problem is that the collecting of vitals and confirmation of medication consumption is not consolidated in one external location for easy access by a professional or loved one. This results in the need to visit the client to collect information to assess how the client is doing.

There is also a lack of control over personalized use of high-risk medications, narcotics and opioids. Using medication tracking applications on personal phones or computers do not sufficiently guide and limit the consumption of these types of addictive and dangerous substances. A solution is required to provide individual client care, but with greater security and reliability to reduce the harm that can be caused by dangerous drugs.

There exists a need for greater control over assignment of medical tracking applications to the intended clients that will use the applications. There is also a need to configure specific actions around medical events and a need to receive the subsequent tracking information that occurs after the medical event is performed by the client. The medical events can include activities such as medication consumption, high-risk drug release, insulin injection, inhaler usage, blood pressure results, weight tracking, insulin readings and others. The tracking mechanism should be performed in a way that requires the client's interaction and involvement to help ensure compliance. Involving the client in the medical act and sending notifications to one or more parties or systems will increase the likelihood that the activity will take place.

### SUMMARY

In accordance with an aspect, there is provided a method for assigning a medical tracking application to a client using an external computer. The method involves: creating a client record with personal and medical elements and storing the client record in a database at an external computer, the client record corresponding to a client; storing one or more client assignment identifiers in one or more databases, the one or more client assignment identifiers corresponding to the client to allow for authentication of a client identity using a medical tracking application running on a medical computer and the external computer, the client identity corresponding to the client; exchanging one or more client assignment identifier messages between the medical tracking application running on the medical computer and the external computer. Upon the client using the medical tracking application, the method involves authenticating the client by processing the one or more client assignment identifier messages to verify the client identity using one or more database lookups of the one or more databases. The method involves codifying, at the external computer, the medical elements from the client record for the client; sending the codified medical elements to the medical tracking application running on the medical computer for execution by the medical tracking application; and receiving, at the external computer, a plurality of medical activities relating to the codified medical elements from the medical tracking application running on the medical computer to allow for generation of alerts for authorized users.

In some embodiments, the one or more client assignment identifiers are securely stored in the medical computer and used for verification after a first verification has been successfully completed.

In some embodiments, the generating of one or more of the client assignment identifiers is implemented at the external computer, uploaded to the external computer or implemented within the medical computer.

In some embodiments, the sending the codified medical elements involves sending only changes that have been made since the codified medical elements had been sent to the medical tracking application running on the medical computer.

In some embodiments, the medical computer running the medical tracking application is one of a personal mobile computer that is property of the client and a shared computer across multiple clients, wherein the multiple clients includes the client.

In some embodiments, each of the one or more client assignment identifiers is one of multiple values composed of one or more alpha-numeric values, one or more biometric signatures or a combination of both.

In some embodiments, the method involves relaying messages, using the external computer as a proxy agent, between the medical tracking application and one or more messaging systems selected from electronic mail, SMS text message, social media messaging and proprietary EMR messaging systems.

In some embodiments, the method involves adjusting, using the external computer, operational parameters from one or more messaging systems selected from electronic mail, SMS text message, social media messaging and proprietary EMR messaging systems.

In some embodiments, the one or more client assignment identifiers include capabilities of the medical computer allowing the external computer to determine whether the capabilities of the medical computer can support requirements of the medical elements.

In some embodiments, the method involves matching the capabilities of the medical computer against the requirements of the medical elements to further authenticate the medical computer capabilities of supporting the medical tracking application to execute the codified medical elements.

In some embodiments, the codified medical elements comprise one or more required actions to fulfil the medical elements, the one or more required actions comprising at least one of biometric collection, barcode scanning, QR-code scanning, RFID communication and weight scale readings.

In some embodiments, the codified medical elements direct the medical computer to use a short-range communication method to release one or more securely held medications being held for the client.

In some embodiments, the codified medical elements indicate health appliances, video equipment and environmental sensors that can be added to the medical computer and timing information for when each of the health appliances, video equipment and environmental sensors should be activated and used.

In some embodiments, the codified medical elements limit client activities, vital readings and environmental factors to raise alerts on the medical computer and to configured users at the external computer.

In some embodiments, the plurality of received medical activities triggers configured notification actions within the external computer, the configured notification actions comprising SMS messages, email messages, EMR signals and social media interactions.

In accordance with an aspect, there is provided a method of managing client computer assignments to clients using an external computer, using one or more client assignment identifiers and a medical tracking application. The method involves: upon locating a medical tracking application on a medical computer, exchanging one or more client assignment identifier messages between the medical tracking application running on the medical computer and an external computer; verifying the client using the one or more client assignment identifier messages. Upon verification of the client, the method involves receiving codified medical elements corresponding to the client from the external computer; executing the codified medical elements such that the medical tracking application: notifies the client when one or more medical elements have come due and at least one encoded action is required; monitors the client performing the at least one required encoded action to indicate completion of the one or more medical elements; and reports on all actions and incorrect actions of the client related to the encoded medical elements.

In some embodiments, receiving codified medical elements involves one or more actions to be performed by the client to confirm completion of necessary steps to fulfil the medical elements, including but not limited to barcode scanning, QR-code scanning, biometric collection, RFID communication, vital sign collection and weight scale readings.

In some embodiments, the incorrect actions include one or more failures to perform an action, the one or more failures including at least one of, performing the action in an incorrect way, performing the action early, performing the action late, performing the action excessively or not performing the action within a required time range.

In some embodiments, the one or more client assignment identifier messages to the external computer indicates that the medical computer used one or more stored client assignment identities to verify the client successfully.

In accordance with an aspect, there is provided a system for assigning a medical tracking application to a client using an external computer. The system includes: at least one memory storing a client record with personal and medical elements and storing the client record in a database at an external computer, the client record corresponding to a client; one or more databases at a medical computer and the external computer for storing one or more client assignment identifiers used for verifying an identity of a client; a medical tracking application on the medical computer for exchanging one or more client assignment identifier messages allowing shared authentication of the client identity using one or more databases within the medical computer and the external computer; and the external computer with at least one processor. The processor is configured for: exchanging one or more client assignment identifier messages to allow for the shared authentication of the client identity using the one or more databases within the external computer and the medical computer; upon detecting that the medical tracking application is running on the medical computer, authenticating the client using the one or more client assignment identifier messages to verify the client identity and codify the medical elements from the client record for the client; sending the codified medical elements to the medical tracking application running on the medical computer for execution by the medical tracking application; and receiving a plurality of medical activities related to the codified medical elements from the medical tracking application running on the medical computer; and wherein the external computer communicates with the medical tracking application running on the medical computer over at least one network.

In an aspect there is provided a method for assigning a medical tracking application to a client using an external computer. The method involving: creating a client record with personal and medical elements and storing the client record in a database at a external computer, the client record corresponding to a client; generating a client assignment identifier that correspond to a client to allow for authentication of the client's identity using a medical tracking application on a medical computer; providing the client assignment identifier; receiving, the client assignment identifier from the medical tracking application running on the medical computer; authenticating the client who is using the medical tracking application running on a mobile device by verifying the client assignment identifier using a database lookup and codifying the medical elements from the client record for the identified client; sending the codified medical elements to the authenticated medical tracking application running on the medical computer for execution by the medical tracking application; and receiving a plurality of medical activities related to the codified medical elements from the authenticated medical tracking application running on the medical computer.

In some embodiments, the receiving and the sending uses one or more secure communication channels between the external computer and medical tracking application or uses one or more secure communications methods.

In some embodiments, the external computer acting as a proxy agent to relay messages between the medical tracking application and one or more messaging systems selected from electronic mail, SMS text message, social media messaging and EMR messaging systems.

In some embodiments, the external computer allowing adjustment of the operational parameters from one or more messaging systems selected from electronic mail, SMS text message, social media messaging and proprietary EMR messaging systems.

In some embodiments, the received client assignment identifier includes the capabilities of the medical computer allowing the external computer to determine whether the capabilities of the medical computer can support requirements of the medical elements.

In some embodiments, the method involves matching the capabilities against the requirements of the medical elements to further authenticate that the medical computer is capable of supporting the medical application's ability to execute the codified medical elements.

In some embodiments, the codified medical elements comprise one or more required actions to fulfil the medical elements, including but not limited to barcode scanning, QR-code scanning, RFID communication and weight scale readings.

In some embodiments, the plurality of received medical activities triggers configured notification actions within the external computer including SMS messages, email messages, EMR signals and social media interactions.

In an aspect there is provided a method of managing client computer assignments to clients using an external computer, using a client assignment identifier and a medical tracking application. The method involving: locating a medical tracking application on a medical computer; providing a client assignment identifier to the medical tracking application running on the medical computer as received from an external computer; processing the client assignment identifier at the medical tracking application and transmitting the client assignment identifier to the external computer; receiving, codified medical elements corresponding to the client from the external computer; executing the codified medical elements such that the medical tracking application: notifies the client when a medical element has come due and an action is required; ensures the client performs the required encoded actions to indicate they have completed the medical elements, and reports on all actions and inaction of the client related to the encoded medial elements.

In some embodiments, the transmitted client assignment identifier comprises capabilities of the medical computer, the method further comprising determining whether the capabilities of the medical computer can support requirements of the medical elements.

In some embodiments, receiving codified medical elements comprises one or more actions to be performed by the client to confirm they have performed the necessary steps to fulfil the medical elements, including but not limited to barcode scanning, QR-code scanning, RFID communication, vital sign collection and weight scale readings.

In some embodiments, the inaction can include failure to perform the action in the required time.

In some embodiments, the action can include performing the action but in an incorrect way or using the wrong medical element.

In an aspect, there is provided a system for assigning a medical tracking application to a client using an external computer. The system involves: at least one memory storing a client record with personal and medical elements and storing the client record in a database at a external computer, the client record corresponding to a client; a medical tracking application on a medical computer for generating a client assignment identifier that correspond to a client to allow for authentication of the client's identity; and an external computer with at least one processor configured for: providing the client assignment identifier; receiving, the client assignment identifier from the medical tracking application running on the medical computer; authenticating the client who is using the medical tracking application running on a mobile device by verifying the client assignment identifier using a database lookup and codifying the medical elements from the client record for the identified client; sending the codified medical elements to the authenticated medical tracking application running on the medical computer for execution by the medical tracking application; and receiving a plurality of medical activities related to the codified medical elements from the authenticated medical tracking application running on the medical computer; wherein the external computer communicates with the medical tracking application running on the medical computer over at least one network.

### BRIEF DESCRIPTION OF DRAWINGS

The following detailed description of the invention is better understood when read in conjunction with the included figures. The included figures are intended to illustrate one implementation of the invention for one skilled in the art. These exemplary illustrations are not intended to limit the disclosure to the specific embodiments shown herein.

FIG. 1 is an illustration of one embodiment where the main elements of the system are illustrated.

FIG. 2 is an illustration of another embodiment where the main elements of the system are illustrated.

FIG. 3 shows an example dataflow chart illustrating the main steps within an external computer to support the proposed process.

FIG. 4 shows an example dataflow chart illustrating the main steps within a medical computer to support the proposed process.

FIG. 5 is an illustration of one example of a configuration screen for configuring various medical elements for a client at an external computer.

FIG. 6 is an illustration of one example assignment screen at an external computer for assigning a medical tracking application to a client.

FIG. 7 is an illustration of another embodiment where example elements of the system are illustrated.

FIG. 8 is an illustration of an embodiment of an artificial intelligence (AI) model that could be used in the medical tracking application to augment traditional software logic and operation procedure.

### DETAILED DESCRIPTION OF DRAWINGS

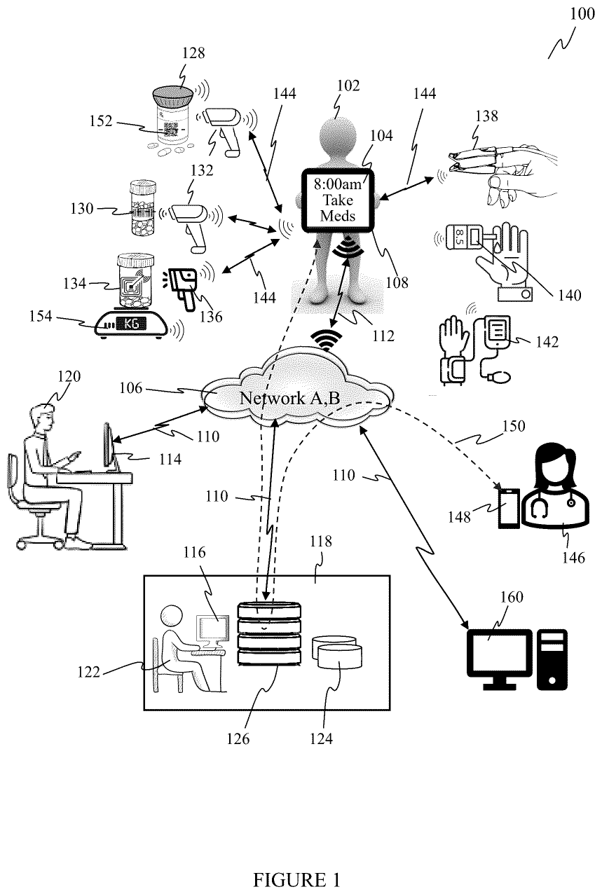

FIG. 1 shows an embodiment of system 100 of the main elements related to assigning medical tracking applications 104 to individual clients 102 using an external computer 118. System 100 creates 114, 116 a client record with personal and medical elements and stores that client record in a database 124 at the external computer 118. The client record is created for the client 102 and corresponds to the client's profile within the external computer 118. Depending on the embodiment, the external computer 118 and the medical tracking application generate and store one or more client assignment identifier messages that can contain one or more alpha-numeric client assignment identifiers, biometric client assignment identifiers and identity confirmation indications. These messages correspond to a client record in one or more databases with the external computer's database 124, and the medical computer's 108 database. The medical computer 108 is a computer device that interacts with the client 102 to provide medical services. The medical computer 108 resides closest to the client 102 to provide the client 102 with messages or notifications indicating guidance, prompts and reminders. Operational directives are provided by the external computer 118 to the medical computer 108. In some embodiments, the medical computer 108 might attach to other medical devices, such as, for example, EKG machines, heart rate monitors 138, blood pressure cuffs 142 and readers, glucose monitors 140, weight scales 154, scanners 132, RFID and NFC devices 136 and the like. In some embodiments, the medical computer 108 can further use external devices, such as, for example, environmental sensors, air quality sensors, cameras and monitoring devices. Additional examples of medical devices are provided in relation to the FIG. 1 description.

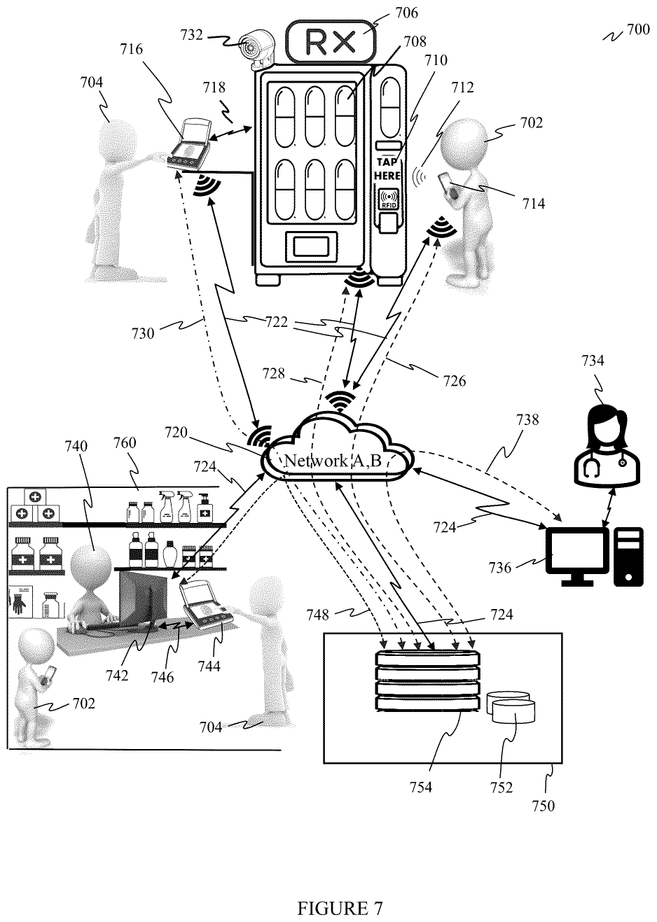

Storage of information at the medical computer 108 would be consider backup storage for the external computer's database 124. In some embodiments, one or more client assignment identifiers might be generated by the external computer 118, stored in the external computer database 124 and used to build client assignment identification messages. In some embodiments, the client assignment identifier could be generated at another computer, shown in FIG. 7 and provided to the external computer in a client assignment identifier message for the client record and then stored in the external computer's database 124. This client assignment identifier will be used later to authenticate the client's identity 102 and provide access to the medical tracking application 104 on a medical computer 108 by the client 102.

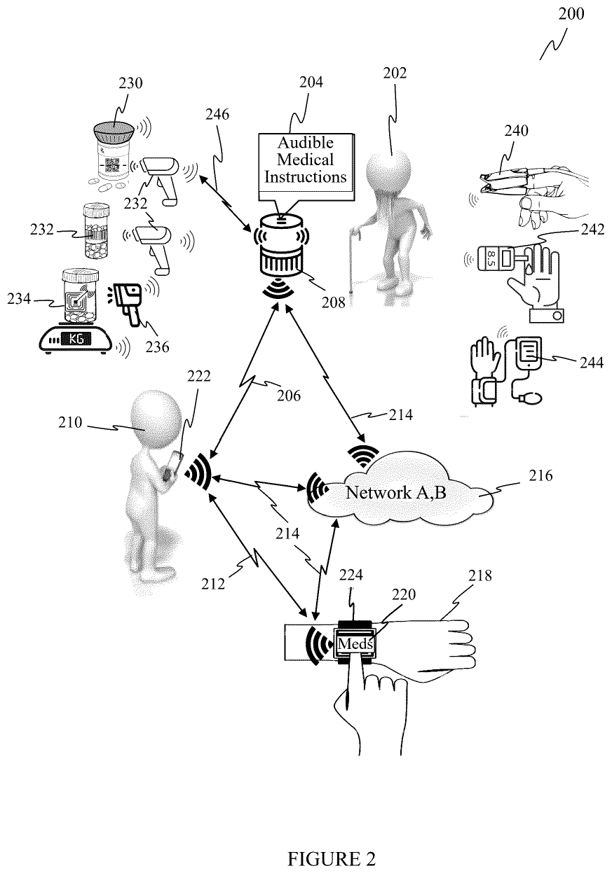

The client record corresponds to a client 102 and is a definition of the client's identity within the external computer 118. The client record includes personal elements and medical elements. Personal elements can include all identities of the client, both alpha-numeric and biometric identities. The client record is formatted to conform to the storage needs and abilities of the external computer 118. When larger cloud computing systems 118 are used, the client record is stored in larger database records 124. In other embodiments where the external computer is a handheld device as in FIG. 2, the client record might be held in one or more files on the external computer. In some embodiments, the personal elements are extensive and can include medical identifications, such as health card numbers, contact details, gender, age, next of kin and home address. In some embodiments, the medical elements can contain current health status, any medication allergy information, medications used and the like. Further details on the client record are found throughout this description.

The medical computer 108 executes the medical tracking application 104. Once authenticated, there are several embodiments for the external computer 118 to transmit the codified medical elements to the medical tracking application 104 for execution. In one embodiment, the entire set of medical elements are encoded and transmitted to the medical tracking application 104. In other embodiments, the current set of previously sent medical elements is known or stored by the medical computer 108 and it is only necessary to send those medical elements that have changed, been updated or added since the last date and time the medical elements had been transmitted. This reduces transmission costs and is more efficient for the processing required within the medical computer 108. In these embodiments, system 100 provides a continuous flow of encoded medical elements every time a change is made to the medical elements on the external computer 118.

The medical computer 108 can be a personal mobile computer that is property of the client 102 or a shared computer shared across many clients, for example. Codified medical elements encompass a wide range of guiding activities, requirements, actions and guidance for the medical tracking application designed specifically for the client 102. In some embodiments, the medical elements can include times, days and frequency of medication consumption across many medications. In other embodiments, the medical elements include how verification takes place before the client 102 can be provided medications from a locked medication holding computer device as described in FIG. 7. In other embodiments, the medical elements can include additional guidance on when to take blood pressure, weight scale, EKG or pulse rate readings. In some embodiments, the codified medical elements include taking glucose readings and matching those readings against a provided scale built for the client 102 to direct how much insulin they should take. An extensive discussion with further examples of codified medical elements is provided later in the FIG. 1 description.

The system 100 sends client assignment identifier messages over various communication methods 110, 112. Depending on the embodiment, these client assignment identifier messages can contain alpha-numeric client assignment identifiers, biometric client assignment identifiers, both types of identifiers and client assignment identifier verification indications. These messages are exchanged between the external computer and the medical tracking application 104 to enable client verification to take place. The client assignment identifier messages can be encoded using many different formats, such as JavaScript Object Notation (JSON) formats, extensible markup language (XML) formats, MQTT, plain text over HTML and the like. In some embodiments, the client assignment identifier messages are encrypted for additional protection. When the client assignment identifier messages contain biometric data, there can be additional encoding methods used as described later in this section.

In some embodiments, client verification happens within the medical computer 108. In other embodiments, the client assignment identifier is transferred in a client assignment identifier message to the external computer 118 so a database 124 lookup can be performed to match the client assignment identifier against all known client assignment identifiers until a match is found. Once confirmation is received that the client assignment identifier is valid, the codified medical elements are transferred to the medical tracking application 104 for execution. In some examples, the codified medical elements comprise one or more required actions to fulfil the medical element, including, but not limited to, biometric collection, barcode scanning, QR-code scanning, RFID communication and weight scale readings.

The codification process takes the configured medical elements from the external server 118 and prepares them for delivery and processing by the medical computer 108. There are various embodiments for how this codification can take place. In one embodiment, each medical element is given an alpha-numeric readable name and is placed into a message for delivery. In some embodiments, the codified medical elements are carried using a message format, such as JavaScript Object Notation (JSON), XML, AMQP, MQTT, plain text over HTML and others. This and many other message formats could be used to carry the encoded medical elements to the medical tracking application on the medical computer 108. In another embodiment, bandwidth or cost concerns are higher, for example, when using Internet of Things (IoT) links, so the codification may use a non-readable binary format where each medical element is coded into bytes or nibbles with specific mnemonic values that are specifically agreed upon and coded by the external computer 118 and the medical tracking application on the medical computer 108.

Once received, the medical tracking application uses the codified medical elements to guide its execution direction. This results in a wide range of behaviours, such as, for example, setting of timers, reminders duration, reminder frequency, actions to be taken upon timers being reached, identity verification steps for gaining access to medications, behaviours after reading glucose levels and many more similar activities. While the medical tracking application is running, all medical activities related to the codified medical elements are sent to the external computer.

### The Client Assignment Identifier

One or more client assignment identifiers are used to verify the client 102 and create a trust relationship with the client 102 using the medical computer 108 and the external computer 118. Aclient assignment identifier can be comprised of multiple values. For example, a client assignment identifier can be comprised of one or more alpha-numeric values, one or more biometric signatures or a combination of both.

There are various embodiments for how a client assignment identifier could be collected and exchanged for use. In one embodiment, the format of the client assignment identifier is a series of letters which could be exchanged using a common medium, such as electronic mail, SMS mobile phone message, a social media message a prompt on a web page and many others. This format might be common when a User Id and Password are used for one of the client assignment identifiers. In another embodiment, the format might be numeric and is a set of numbers. This format might be common if a PIN value is used as the client assignment identifier. In another embodiment, the value is a non-readable digital value, potentially using an encryption method for exchanging the value. In this example, the value might have to be cut and copied into the medical tracking application in order to verify the client's 102 identity. In another embodiment, a biometric value is used and exchanged.

There are several embodiments for how the client assignment identifier is generated, stored and verified. The client assignment identifier corresponds to a client's medical information and therefore must be matched before the medical tracking application 104 can proceed with operation. Since no medical elements are added at the medical tracking application 104, it is important that the external computer 118 provide all this information. Client assignment identifiers are exchanged in client assignment identifier messages. In those embodiments where the medical computer 108 has stored all essential alpha-numeric client assignment identifiers and any biometric client assignment identifiers, the client assignment identifier message can also contain just confirmations that the client is verified.

In some embodiments, client assignment identifier messages can include the capabilities of the medical computer 108. These capabilities allow the external computer 118 to determine whether the capabilities of the medical computer 108 can support requirements of the medical elements. There are various ways the required capabilities can be built and added to the client record at the external computer 118. In one embodiment, they are automatically added when different medical requirements are requested. For example, the use of a Bluetooth scanner will require the medical computer 108 to have Bluetooth support. The use of a RFID or NFC device will require the medical computer 108 to have RFID or NFC support, respectively. If photographic proof of consumption is required in the medical elements, the medical computer 108 may have a built-in camera. When the client assignment message contains some capabilities, it gives the external computer 118 the option of verification.

In one embodiment the client assignment identifier is generated at an external location, as highlighted in FIG. 7, like a hospital, a pharmacy, a drug clinic, a safe consumption site or similar monitored location. After showing photo ID of themselves, the client might provide a biometric identity with the supervision of a professional. This identity is then communicated to the external computer 118 in a client assignment identifier message, where it is then stored in the database 124. In one embodiment the saved client assignment identifier is verified at the external computer 118 when it is entered by a client. In another embodiment it is relayed in a client assignment identifier message to the medical tracking application 104 from the external computer 118 to perform a local verification of the client. Biometric identity can also refer to encoding and compressing methods for exchanging biometric data between systems. Methods like Common Biometric Exchange Format (CBEFF) using a Biometric Information Record (BIR) might be used, as well as compression methods like wavelet scalar quantization (WSQ) and JPEG 2000 might also assist with exchange and processing of biometric data, to name just a few techniques.

In other embodiments, the client assignment identifier is generated at the external computer 118 and stored in the database 124. In some embodiments, the client assignment identifier is a User ID and password, easily shared and provided to the medical tracking application 104 in a client assignment identifier message. In other embodiments, the client assignment identifier could be a shorter one-time PIN value that is used once to assign the medical tracking application 104 to the client until the external computer 118 terminates the assignment.

When using a User ID and password or PIN code, the client assignment identifier is generated on the external computer and is provided in a client assignment identifier message to the client 102 or the client's agent or caregiver with authorization from the client. The client assignment identifier is then received in a client assignment identifier message by the external computer 118 from the medical tracking application 104 running on a medical computer 08. This allows the external computer 118 to authenticate the client 102 using the medical tracking application 104 running on a medical computer 108. In some embodiments the medical computer could be the client's own personal medical computer 108. For example, a personal tablet, cell phone, watch or smart speaker. In some embodiments the medical computer 108 might be provided by a professional like a doctor 120, pharmacist 120 or nurse 146. The medical computer 108 could also be provided by an organization 118, 160, like a hospital 118, 160 to use with a hospital-at-home type support service.

In other embodiments, the medical computer 108 might be shared between people. In one embodiment, an elderly husband and wife share a single medical computer 108 and enter their biometrics, such as a fingerprint or facial recognition, to gain access to the medical tracking application 104. Once authenticated the medical tracking application 104 loads the medical elements for that individual to guide them on what should be happening with their health care. In other embodiments the medical computer could be shared across strangers for safe dispensing of high-risk medications like opioids. Each client would have a PIN and biometric that would need verification before any medication would be released to them.

Entering the client assignment identifier into the medical computer 108 might also be augmented with biometric input, like a fingerprint scan, facial recognition, palm scan, retinal scan or others. This adds an additional layer of protection to further confirm the client 102 using the medical computer 108 is the same person 102 each time.

Verification of the client assignment identifier can be done in several ways. In one embodiment, verification is performed at the external computer 118. This is done by performing a lookup in database 124 to verify a match for the client assignment identifier received to an identifier saved in the database 124. Once verified, the medical elements from the client's record are codified for the identified client 102. These codified medical elements are then sent to the authenticated medical tracking application 104 running on the medical computer 108 for execution by the medical tracking application 104. All medical activities generated by the medical tracking application 104, that are related to the codified medical elements, are then sent to the external computer 118 from the medical application running 104 on the medical computer 108.

In another embodiment the external computer 118 might delivery client assignment identifiers in a client assignment identifier message to the medical computer 108, after it has saved it in the database 124. For example, if a kiosk-based operation is being used the client 102 might walk up to the medical computer 108 and enter their personal PIN. This PIN is then sent to the external computer 118 and the corresponding client assignment identifier, for example a biometric signature, gets sent to the medical computer 108 for verification. The medical computer 108 can then cache a set of these client assignment identities for those clients 102 that visit that kiosk. In another embodiment the biometric might be initially verified at the external computer 118 but then it is provided to the medical computer 108 for any subsequent verification requirements. These embodiments are depicted in detail in FIG. 7.

In some embodiments, the medical computer 108 has saved all elements of the one or more client assignment identifiers within its local memory and database, which can be, for example, a secure storage, sometimes referred to as a secure enclave or secure key storage area. The medical tracking application 104 may have stored this information the first time it was used and potentially received from the external computer 118. In such embodiments, the client assignment identifier message sent to the external computer 118 would be an indication that the client verification was successful. In such embodiments, the reception of the client verification message from the medical computer 108 indicates to the external computer 118 that encoding of the medical elements can take place. In one embodiment, the entire set of medical elements are encoded and transmitted to the medical tracking application 104. In other embodiments, the current set of previously sent medical elements is known or stored by the medical computer 108 and it is only necessary to send those medical elements that have changed, been updated, deleted or added since the last date and time the medical elements had been transmitted. This reduces transmission costs and is more efficient for the processing required within the medical computer 108. In these embodiments, system 100 provides a continuous flow of encoded medical elements every time a change is made to the medical elements on the external computer 118.

### The External Computer

The external computer 118 has a processor 126, a memory 124, one or more local interfaces 116 and supports one or more communication methods 110 to exchange information with other computers. In some embodiments, the external computer 118 can support external computer 114 over a communication method 110. For example, an Internet browser running on a myriad of computers could connect 110 with the external computer 118 over a shared network 106. In some embodiments the external computer 118 can be a personal device such as a cell phone, a tablet or a desktop computer, this embodiment is highlighted in FIG. 2. In other embodiments, the external computer 118 can also be part of a cloud computing environment 118, a private computing facility 118 or a specific company's data center 118.

The external computer 118 has control over the medication tracking application 104 running on the medical computer 108 and directs the actions of the medical tracking application 104 being used by the client 102 to remind, track and manage medication events. In some embodiments, the client 102, while using the medical tracking application 104, could have very limited control over the behaviour of the medical tracking application 104. In an embodiment, the client 102 might be able to adjust a reminder time by ‘N’ minutes to suit their personal preference. Such restrictions would be pre-established and guided by the external computer 118.

The external computer 118 provides various interfaces to an authorized person 120, 122, 146 to configure the client 102 and their health parameters. The goal of the authorized person 120, 122, 146 is to build a configuration for the client 102 that will support their health needs. This authorized person 120, 122, 146 could be a professional, such as a doctor, nurse, PSW, pharmacist, medical professional or someone else known to the client 102. The authorized person 120, 122, 146 has permission from the client 102 to help guide and protect their health needs. For example, the authorized person 120, 122, 146 could be guiding a client who is an elder parent 102. In some embodiments, the authorized person 120, 122, 146 would be working with some advanced skills in health tracking and has an authorized login and password to the external computer 118. In some embodiments, as highlighted in FIG. 2, the authorized person 120, 122, 146 might be authenticated to their own medical computer. For example, the medical computer can be a cell phone, tablet or laptop computer.

There are different environments 114, 118, 148 where an authorized person 120, 122, 146 is performing their support and setup work on behalf of the client 102. In one embodiment the authorized person 120 is working at a remote location using an Internet browser type environment 114 and communicating over a wide-area link 110 using Ethernet and protocols like TCP/IP to exchange information with the external computer 126. These are real-time data exchanges with sub-second interactions to setup information for the client 102. For example, this authorized person 120 could be the client's 102 personal pharmacist, loved one or caregiver 120 who is setting up a drug regimen 104 for tracking medication consumption by the client 102.

In another embodiment the authorized person 122 is using an inter-network computer 116 to access the client's 102 database 124 records to create, modify and track what is happening with the client 102. This connection might use a local area network (LAN) connection behind all firewall and available only to local personnel 122. These are real-time screens and interactions with the client's 102 record for setup, monitoring and tracking purposes. For example, this could even be a health call center 118 that runs its own data center 118 for supporting elderly clients 102. This could also be a senior's home 118 or long-term care home 118 that has set up the system specifically to support those clients 102 that live within the walls of their facility.

In another embodiment a support person 146 is more a mobile professional that might use a messaging passing mechanism 150 to support the client 102. This is discussed in detail in later paragraphs of this figure. This support person 146 can build messages and send them via email messages 150, SMS text messages 150, social media messages 150, EMR private messages 150 or some other message type into the external computer 118. This is not a real-time mechanism and would be best used to adjust and change key configuration parameters for the client 102. However, for a busy individual like a doctor 146, a nurse 146, a PSW 146, a specialist 146, a nurse practitioner 146 or some other professional that might travel between hospitals and their office it is a very quick and efficient way to manage configuration changes to the client's 102 database 124 record at the external computer 118.

The authorized person 120, 122, 146 will establish various personal and medical elements that will be used to support the various medical needs of the client 102. There will be personal information about the client 102 added to the database 124 and medical elements added to the database 124 as well. The interfaces 114, 116 in FIG. 1 could be web browser interfaces using standard HTML and advanced programming methods. The interfaces 114, 116 could also be a direct console interface 116 within a virtual private network (VPN) environment within a company's private data center 118. In most embodiments shown in FIG. 1, these are authenticated connection points 114, 116 where login, passwords and two-factor authentication are being used. FIG. 2 provides other embodiments that will be explored in greater detail.

The external computer 118 must be able to support one or more data communications methods 110, 112 to communicate with the medical computer 108. These are necessary to reach the medical computer 108 and for supporting remote configuration interfaces 114 used for configuration and control purposes. In some embodiments there could be multiple communications methods supported, to allow for the possibility that should one method fail another one can be used as a backup. These might include fixed lines like Ethernet connections, wireless links such as 802.11 (branded Wi-Fi), wide-area cellular data links such as GSM, Edge and 5G technologies and the protocols used to delivery messages over these connections. In some embodiments, the connections are made through one or more networks 106 that are used to deliver messages between the external computer 118 and the medical computer 108. Additional embodiments for the communications link 110 are provided in FIG. 2.

In some embodiments, information is exchanged over the link 110 using a secure communications method. This could involve one or more methods of streaming encryption, elemental encryption, scrambling or other security methods. In some embodiments, pre-existing encryption between any two endpoints over a network 106, like the Internet 106 that uses protocols like SSL, TLS, SSH HTTPS and others that can be enabled to ensure the privacy of the information. In these environments, there could also be encryption certificates used to verify tokens, passwords and identities. In some embodiments, secure channels might also be augmented with additional encryption of specific elements or parts of the message. In some embodiments, the entire message contents are encrypted using a private encryption key before being sent over a secure channel. The external computer 118 and the medical tracking application 104 might exchange a private symmetrical key used with an encryption algorithm like triple DES.

In some embodiments, the client assignment identifier is used to provide a seed value for a negotiated shared encryption key. By using a protocol such as simple password exponential key exchange (SPEKE), a well known Diffie-Hellman key generation method can be augmented. This augmentation of Diffie-Hellman can protect key negotiations from the well-known man-in-the-middle attack that has made Diffie-Hellman unsuitable for key negotiations. In some embodiments, quantum encryption methods that use advanced techniques can be used to stop future quantum computers from decrypting data that should by private.

Another advantage of the external computer 118 in FIG. 1 is that it can provide proxy services 150 to external users 146 to the system. In some embodiments, professionals 146 such as doctors, nurses, personal support workers (PSWs), nurse practitioners and other health care individuals might want a rapid method to interface with a client 102 who is under their care. Many times, these individuals 146 do not have the time to login to advanced interfaces 114 but need an efficient and improved method to perform an action for their client 102. In these embodiments, the professional 146 has a cell phone, tablet or laptop available 148 and needs a solution that is fast, secure and safe. There are several embodiments where such a medical proxy service 150 could be of use to help these professionals 146.

In some embodiments, during the onboarding process, the professional 146 is given a private link, similar to an URL, that can be used as a security token to connect to a client's 102 configuration and messaging center later. In some embodiment the security token is a private URL that can be used to reference this client's 102 database 124 information and messaging subsystem. The professional 146 would be provided several actions they can perform around configuration of the client 102 and messaging to the client 102.

The onboarding process involves qualifying authorized people 120, 122, 146 to ensure their contact information is correct and to allow them to accept the role the client 102 has authorized them to do. For example, if email address and cell phone numbers have been provided it is a chance to send a message to these addresses and phone numbers to confirm a typing mistake has not taken place. Usually, a temporary URL is provided that takes the authorized person 120, 122, 146 to a web page where they must perform a legally binding act to accept the responsibility of supporting the client 102.

The onboarding step can take place during the initial creating the client and configuring their caregivers. This is the stage where healthcare professionals 146, caregivers 120 and others 122 are defined and related directly to the client 102. In most embodiments, the client 102 provides the name of the professional 146 to the medical tracking application 104 and then the professional 146 receives a message from the client device 108 to confirm the client's 102 wishes and to perform a legal acceptance of the role of caregiver. Once the professional 146 is fully onboarded, the external computer 118 can send a final message using the agreed messaging technology. In some embodiments, the proxy service 150 is provided via a private messaging system 150 used by an electronic medical record (EMR) system 150 used by many healthcare professionals 146. In some embodiments, the proxy message exchange 150 might use a text message, an email message, or a social media message to send the private link and exchange all messages 150.

In some embodiments, the client 102 can send a private message from the medical tracking application 104 to the external computer 118 to connect to a professional 146. The external computer 118 proxies the client's 102 message into an email or text message 150 through the external computer 118 to enable a two-way communication 150 with the health care professional 146. A message originating from the medical computer 108 and the medical tracking application 104 extends a trust umbrella to the professional 146 and allows future messages to be received and passed along from that professional 146 only.

The proxy service 150 offered by the external computer 118 will vary based on each embodiment the solution is used in and the needs of the system. In some embodiments, the proxy message 150 is focused on the exchange of messages 150 with the client 102. These messages may range from simple messages such as ‘how are you doing’ to more important and detailed messages such as ‘please take your blood pressure now and send me the results’. Similarly, the client 102 can reach out to the professional 146 to create a stronger bond with that person 146, with a result of overall improved health monitoring.

In some embodiments, the proxy service offers a set of commands 150 that affects the configuration for the client 102. For example, the message 150 might include a syntax like: ‘Morning Dose Time: 10:30 AM’. Sending this message with the private link for the client 102 results in the medical tracking application 104 setting a morning dose reminder time for 10:30 AM for that client 102. Such a command 150 might affect medication dose reminder times, times used to request important vitals to be taken, medication names, dose strengths and other dynamic changes in the system that only a health care professional 146 might be aware of.

In some embodiments, the professional 146 might request that all medical device 132, 136, 138, 140, 142 readings that are taken by the client 102 be relayed directly to them using a defined method. For example, this might result in blood pressure 142 readings, or glucose readings 140 being send via SMS text message to the client's 102 doctor 146 in near real-time. This would be simple for the doctor to manage and provide a level of care that might save lives.

Proxy services further include the ability to allow health related health devices 138, 140, 142 to be added dynamically and monitored. For example, if a client 102 is one day prescribed a heart medication a doctor 146 might want to add support for a specific blood pressure cuff 142 and establish times to perform vitals readings. This support by the external computer 118 could be done remotely via the professional 146, or directly while sitting 122 at the external computer 118. This does assume the client computer 104 supports the capabilities to support the health device. Should the client computer 104 fail to offer that level of technical capability, the requestor 120, 122, 146 would be given an error message by the external computer 118.

By offering control over the addition of external health devices 138, 140, 142 the level of control over the type and quality of the health device 138, 140, 142 is known and guaranteed to meet a certain standard so the vitals results can be trusted. Further external services could also be added to the client's computer 104. In some embodiments the environmental conditions of the client 102 are extremely important to monitor. There might be environmental sensors that can be connected via a command from the external computer 118 as triggered by a professional 120, 122, 146. Monitoring environmental variables like temperature, air quality, CO and smoke levels could help in many cases for elderly clients 102. Using an environmental sensor as a health device is a key method to extend the variables that can be considered by a health professional.

The medical elements within the external computer 118 also support artificial intelligence (AI) behaviors within the medical tracking application 104 running on the medical computer 108. In one embodiment, as information is collected from a large population of individuals, it might become clear that a certain aged person, of a certain gender will usually fail to take their medications before bedtime. This tendency might be due to tiredness, poor planning or forgetfulness as elderly people often are more forgetful at the twilight hour. An AI component could be developed to watch for this tendency and suggest earlier times on certain days, or every evening when an elder male client 102 must take medication before bed. In another example, an AI component could be configured to collect large samples of data related to heart conditions and heart medications. In this embodiment, it could be likely that a certain population of people might require their heart medication 20 minutes sooner than normally prescribed to maximize the best effectiveness of the drug and reduce the chance of a major heart incident, such as, for example, a heart attack, arrhythmia events, stroke potential, angina attacks and atrial fibrillation. Often, the timing of medication is critical for each person and with an AI component analyzing different blood pressure readings before and after medications are taken, optimization patterns can be established and reminder times with improved accuracy can be developed and refined.

The medical elements provide guidelines and limits to parameters in complex and varied ways to enable artificial intelligence decision-making by the medical tracking application 104. In some embodiments, the medical elements include behavioural elements for when actions are taken or not taken. Some embodiments include limits on vitals readings for being too high and too low in various categories. Other embodiments further include limits on external environmental variables to guide what is safe or not as safe for a given client 102. In other embodiments, the AI components run at the external computer 118, which feeds updated medical element information to the medical tracking application 104 to improve its behaviour and operation.

For example, if a client is on a COPD medication and they are of a certain age and gender, they must take their medication with ‘N’ minutes of the recommended time. If they fail to do this and the environmental parameters indicate a slightly elevated inside temperature with excessively high humidity then an alarm could be raised on the medical computer 108 and sent back to the external computer 118 to alert health care professionals 146, family and caregivers.

The external computer 118 also has links to other supporting computer systems 160. These supporting computer systems 160 can include a wide range of services and interface systems 160. In some embodiments when the external computer 118 receives a medical activity event, it triggers notification actions with one or more supporting computer systems 160. This can include triggers around medication usage, failure to use and incorrect use of medication consumption by the client 102. The triggers can also include reminders to take vital signs, the failure to take vital signs when requested and the failure to take any vital signs. The external computer 118 might use email servers 160, third-party SMS Text message services 160, IoT cloud services 160 and social media connections 160 to send important messages and alerts to the client 102, the client's 102 caregivers 146, the client's 102 professional support staff such as doctors 146, pharmacists 146, nurses 146 and PSWs 146 and many others.

In some embodiments, the external computer 118 has connections to supporting computer systems 160 for integration purposes. For example, links to hospital electronic medical record (EMR) systems 160 can allow for a seamless setup of the client's 102 profile within the external computer's 118 database 124. Similarly, an integration to a pharmacy management system (PMS) 160 can streamline information regarding medications to be consumed and their required daily regimen.

### Establishing a Client

The step of creating the client's 102 identity profile on the external computer 118 is a key step in the process of eventually controlling their medical tracking application 104 on the computer system 108 of their choice. Depending on the embodiment, the client's identity profile 102 can have very little information or quite a lot of information about the client 102. In some embodiments, a name may be the only information provided to identify the client 102. In some embodiments, the client's 102 identity profile has a name, one or more assigned health care numbers from their state, province or country, their contact information, cell phone, email address, every caregiver 146 that they wish to be monitored by, notification preferences and other settings. These are saved in the database 124 and will be used later to build functionality into the external computer 118 to support the client's 102 medical needs.

During the process of creating the client 102 profile, it is also possible to define medical elements for the client 102. These medical elements can be defined before assignment to a medical tracking application 104, or they can be defined after the assignment to a medical tracking application 104. The medical elements reside alongside the client's 102 database 124 record. Having these elements permanently in the external computer's 118 database 124 allows the assignment and re-assignment of the client 102 to many physical medical computers 108. For example, if the medical computer 108 were to fail, be stolen or have a minor issue, it could be unassigned and the client 102 can then re-assigned to a new computer 108 very quickly with all the same information taking effect the moment the assignment is confirmed when the medical tracking application 104 sends the client assignment identifier.

Medical elements can range very dramatically based on the nature of the medical system and what is required for it to track. In some embodiments, medical elements might focus on collecting vital sign information. Using various devices 138, 140, 142, the medical tracking application 104 might remind the user and collect vital sign information from the range of devices 138, 140, 142. These devices 138, 140, 142 may support Bluetooth, Wi-Fi, RFID or proprietary wireless protocols for direct communication with the medical computer 108. As a client's 102 health changes these medical devices 138, 140, 142 can be added and removed as needed. This can be done directly or via a proxy method using SMS, Email or similar communication messages.

Medical elements can also provide advanced behaviours that can be followed by the medical tracking application 104 for the various connected medical devices 138, 140, 142 connected to the medical computer 108. In one embodiment the medical elements contain a glucose range for a given client 102. Receiving a reading from the blood glucose meter (glucometer) 140 that was outside of a given client's 102 safe glucose range would instantly raise an alarm for the client 102 to administer insulin supporting medications. Since each client's 102 optimal glucose range can vary using the external computer 118 and the medical elements to guide the range is an ideal way to ensure a customized behaviour for each client 102.

In another embodiment the medical elements might contain a weight range for a given client 102. If a client 102 with high blood pressure or some other condition gets on a weight scale and the reading that is received into the medical tracking application 104 is outside of the prescribed weight, then a notification is raised suggesting the client 102 should take a diuretic or some other form of medication supports. These reminders might also be supported by a questionnaire asking the client 102 to select a pain scale or discomfort scale. This information could be relayed to the external computer 118 and shared with all authorized support personnel 120, 146.

In another embodiment, the medical elements might indicate that a blood test is required for an AIDS patient. This test might be done at a health center, or the client 102 might have a portable blood testing device connected to the medical computer 108. If the test is not performed on day 1 a local notification is raised to remind the client 102. If the blood test is not performed on day 2 another stronger notification is raised but a message is sent to the external computer 118 to be relayed to all support medical personnel 120, 146. These and other advanced behaviours are possible when using the medical elements. The ability to customize the medical elements for every client 102 creates a perfect and detailed mechanism to support complex health needs in a real-time way.

In some embodiments, medical elements can also include the required capabilities of the medical computer 108. In some embodiments, these requirements might include a range of physical capabilities to allow specific actions to be taken. For example, the computer 108 might have to be capable of certain biometric functions like fingerprint reading or facial scans. The computer 108 might have to be at a certain hardware revision with a camera capable of barcode and QR code scanning. The computer 108 might require near field communication (NFC) capabilities, a subset of RFID functionality. These and other capabilities might be included in the medical elements' configuration step.

Capabilities might further require the ability to connect to external home systems. These systems might include thermostats, smoke alarms, air quality sensors, CO detectors and other environmental conditions.

In some embodiments, the medical elements might include medication tracking requirements. Many potential solutions might be used to track medication consumption using external devices 132, 136. In some embodiments, the medical tracking application 104 performs its tracking based on input proved by these devices 132, 136. More detail will be provided on this part of the solution, but these external devices 132, 136 can provide barcode information 130, QR code information 152, RFID and Near Field Communication (NFC) information 134, as well as weight information 152 and other types of information to the medical tracking application 104 for verification.

In some embodiments, the medical elements can include actions that should be taken when medical tracking information is received from the medical tracking application 104 into the external computer 118, based on the client's 102 actions. In these embodiments, the external computer 118 supports a wide range of configurable notifications that relate to configured actions, that the client should be taking on their computer 108 and detected by the medical tracking application 104. The external computer 118 can set notification triggers on medication events, vital sign requirements and many other medical events.

With the client 102 defined and, optionally, their medical elements defined, a potential client 102 assignment to a medical tracking application 104, running on a computer system 108 can take place. In most embodiments, this step needs to be as dynamic and flexible as the type and range of potential computers 108 that could be selected and used. To support this assignment, the external computer 118 generates a client assignment identifier. This client assignment identifier corresponds to the client's 102 record in the database 124. This client assignment identifier will be used later to authenticate the client's identity 102 using a medical tracking application 104 on the medical computer 108.

In some embodiments, the client 102 identifier is a login and password that can be used to login to the medical tracking application 104. In some embodiments, this provides a permanently assigned login name and password that can be used as needed by the client 102 or their caregiver 146. In some embodiments, one or both of the login name and password might have a time limit before they expire if not used. In some embodiments, the client assignment identifier could be a one-time assignment code that has a short life expectancy. In this embodiment, the medical tracking application 104 receives the client's 102 identity, verifies it once and only once and personalizes itself to that client 102 until an un-assignment occurs. This embodiment might be more common for elderly clients 102 that do not have the medical or physical capabilities to be entering login names and passwords into a device repeatedly.

In other embodiments, the client assignment identifier can be a biometric reading. These embodiments are highlighted in FIG. 7. In some embodiments, the client assignment identifier could be both a PIN and a biometric. Using the PIN might lead to a first identification but then the biometric confirms the PIN is being used by the right person, since a PIN is easily shared. For high-risk medications, such as opioids or opioid agonist therapy (OAT) medications such as methadone and naloxone. In some embodiments, the biometric reading is collected in the medical computer 108, in other embodiments, it is collected remotely and eventually makes it way to the medical computer 108.

When the client assignment identifier is an alpha-numeric value, it can be provided to the client 102 using a variety of methods. In some embodiments, an electronic method is used, for example, an email message, an SMS text message, a social networking message, a secure message exchange platform and other electronic methods. In some embodiments, the client 102 assignment identifier is sent via manual post and can be handed to the client 102 physically at the doctor's office or the pharmacy. In some embodiments, the client assignment identifier could be read off a website or even spoken to the client 102 over the phone. The client assignment identifier is used to identify the client 102 specifically and whether it is a single value, a login name and password, a PIN value, or some other type of legible or illegible value, it should be provided to the medical tracking application 104 to authorize the application's 104 use on behalf of the client 102.

In some embodiments the PIN might already exist and is being used in another medical system. For example, some pharmacy systems like Pharma Net used through the province of British Columbia has a PIN value that can be assigned to every patient in the province. In this case the PIN already exists and a healthcare professional 120, 146 might then insert that value in the database 124 of the external computer 118. In this embodiment there would be less of a need to transmit the PIN to the client 102 as they already know their own PIN. This might also be another reason to use a biometric confirmation method as the PIN might also be known by many people if it has been used for many years.

### The Medical Computer

The medical computer 108 represents one of the endpoints for communications with the external computer 118. The medical computer 108 provides one or more interfaces to the client 102 for input events, notification events and communications with other computers both locally and remotely. The medical computer 108 forms an authenticated relationship with the external computer 118 via the medical tracking application 104. One of the goals of the external computer 118 is to manage medical computers 108 by exchanging and verifying client assignment identifiers using client assignment identifier messages from medical tracking applications 104.

The medical computer 108 can be any virtual computing device 108 that can run a computer program. For example, this might include desktop computers, laptop computers, mobile computers such as tablet computers, cell phones and wearable computers like watches, glasses and others. It might include custom computers with specialized interfaces for biometric collection, like facial recognition and fingerprint scanning. Each of these computing devices 108 will have properties and characteristics, and these must be able to support the required capabilities if specified within the client's 102 profile on the external computer 118. In many embodiments, a mobile computer 108 supports the ability to travel to where the client 102 needs to be in a given day. This mobility can be valuable to help the client 102 stay on top of medical needs and optimum health for their age and stage of life.

In some embodiments the process starts within the medical computer 108 by locating a medical tracking application 104 on the medical computer 108. In some embodiments, locating can involve navigating on the medical computer's 108 interface to find a pre-loaded medical tracking application 104. In some embodiments, the client 102 must first download the medical tracking application 104 from an App Store or as provided by the solution provider onto the medical computer 108. In some embodiments the medical computer 108 is a dedicated computer 108 that is designed and linked to other systems to perform a single task. FIG. 7 highlights some of these embodiments.

Once the medical tracking application 104 is running, the medical tracking application 104 begins verifying the client 102. In some embodiments the medical tracking application 104 is always running and once authenticated previously remains always available to the client 102. In some embodiments the client 102 starts the process of verification by providing one or more client assignment identifiers. These can be alpha-numeric, biometric or both kinds of client assignment identifiers. As mentioned, alpha-numeric client assignment identifiers could have been received via email, SMS, or social media messages, read and copied down off a website page, verbally shared over the phone, received within a letter from the mail or many other ways. The verification can result in several client assignment identifier messages being exchanged with the external computer 118 depending on the embodiment and where the various one or more client assignment identifiers are stored.

The medical tracking application 104 goes through the client assignment identifier verification process with various interactions with the external computer 118. From the client's 102 perspective, only when the verification process is completed will the medical tracking application 104 receive the corresponding codified medical elements.

Once codified medical elements or any updates to the medical elements are received from the external computer 118, the medical tracking application 104 will then execute the codified medical elements on behalf of the client 102. This allows the medical tracking application 104 to notify the client 102 when any medical element is due, or an action is required. It can also ensure the client 102 performs the required encoded actions to indicate they have completed the medical elements. The medical tracking application 104 then reports on all actions and inactions of the client 102 related to the encoded medial elements.

### The Medical Tracking Application

The medical tracking application 104 is the connection point to the external computer 118 for a given client 102. Once authenticated, the medical tracking application 104 and the external computer 118 have a trusted relationship. The trusted relationship extends to the client 102 who has been authorized to use the medical tracking application 104. The trust is built upon the exchange of one or more client assignment identifier messages with the external computer 118 for verification. This verification is performed by one or more database 124, 108 lookups within the external computer 118 or the medical computer 108. Once the one or more client assignment identifiers match known client 102 values already created within the one or more databases 124, 108 the verification process is complete.

In some embodiments, when the medical tracking application 104 sends the client assignment identifier message, it includes the capabilities of the medical computer 108. For example, this might include biometric capabilities, communication options like Bluetooth and Wi-Fi, camera abilities, hardware and software version codes, NFC abilities and other functional capabilities. When these capabilities are received with the client assignment identifier, the external computer 118 has the ability to match the capabilities to the configured required capabilities. If there is a match, or if the computer 108 has more capabilities than required, the assignment process can proceed. Otherwise, the assignment process could be terminated and the client 102 will need another medical computer 108.

In some embodiments, the trusted relationship can be re-enforced with security, encryption and additional authentication steps like two-factor authentication. In some embodiments, the client 102 authenticates themselves to the medical computer 108 being used by providing a biometric such as a thumbprint, a matching facial scan or a PIN code that the medical computer 108 is expecting to identify the medical computer's 108 owner. In other embodiments, the external computer 118 requests two-factor authentication of the client 102 by sending periodic verification requests via SMS text messages or emails to their cell phone or email account, respectively.

As mentioned previously, the medical tracking application 104 can be developed and ran on a wide variety of medical computers 108. This might include cell phones, tablet computers, laptop computers, worn devices like watches and desktop computers. FIG. 2 details some of the embodiments around other form factors, especially involving worn devices like watches, computer glasses, smart speakers and other types of worn computers. The medical computer 108 offers a character input ability and is able to output different notifications. In some embodiments, this might be a sound reminder, a visual colour reminder, a flashing icon reminder or a combination of several different types of output notifications. The medical tracking application 104 requires from the medical computer 108 a data communication capability to reach the external computer 118 using one or more data communication methods and protocols. This might involve one or more wired or wireless networks 106. Network technologies might include Bluetooth, NFC, Wi-Fi, GSM protocols and even proprietary wireless protocols and connection methods.

In some embodiments, the medical tracking application 104 does more than just remind the client 102 about medical events that need to happen. In these embodiments, the medical tracking application 104 might require action and collect input from the client's 102 actions. In some embodiments, the medical tracking application 104 will suggest follow-up actions be performed based on input from prior actions. In some embodiments, the notification subsystem will not stop notifying the client 102 until the action is completed. In this way the client 102 is forced to be involved in the medical event, which has been shown to increase the likelihood that the event will take place. The following is a small list of examples of the types of interactions the medical tracking application 104 can have with external devices:
A medication 152 must be taken at 9:00 am, so the client 102 must use a scanner 132 or the medical computer's 108 camera to scan a QR code 152 to confirm they are holding the correct medication 152. They screw off the lid 128 and the lid 128 detects it has been removed, and the contents of the medication container have been exposed. The lid 128 also supports a communications protocol such as Bluetooth 144 and reports the change in status.
A medication 130 is due, so a scanner 132 or the medical computer's 108 camera must be used to scan a barcode 130 to confirm the correct drugs are about to be taken.
A medication 134 needs to be taken, so an RFID (NFC) reader or the computer's NFC capability is used to detect the correct medication 134 has been located by the client 102. They must also weigh 154 the medication container 134 after removing a pill for consumption to confirm they have indeed taken the correct amount of medication out of the container 134. The weight scale 154 supports a communications protocol like Bluetooth or NFC 144 that communicates all state changes to the medical tracking application 104.
A client 102 must take their pulse rate and put on a finger pulse monitor 138 that communicates to the medical tracking application 104 the result. Until this action is performed, a notification is played to encourage the client 102 to perform the action.
A client's 102 glucose levels must be read and recorded to check their insulin level. Using a Bluetooth-enabled 144 glucose reader 140, the current level is sent to the medical tracking application 104 and passed onto the external computer 118 to record for tracking purposes. In some embodiments, this reading 150 is relayed onto the medical professional 146 for immediate review.
A client's 102 glucose levels are read via a Bluetooth-enabled 144 glycosometer 140 and the level received is compared to a chart provided via the medical elements. Based on the reading the medical tracking application 104 suggests a level of insulin that is required to be consumed or injected by the client 102.
A client's 102 blood pressure reading might be required at a certain time. Until the blood pressure cuff 142 is used, the medical tracking application 104 persists in reminding the client 102 that this step must be performed.
The medical tracking application 104 runs a timer and every hour it takes interacts via Bluetooth to a home environmental sensor. It reads the current temperature and air quality and sends a message containing this information to the external computer 118.
The artificial intelligence component within the medical tracking application 104 detects an air quality and humidity issues for a patent with COPD breathing issues. They are 30 minutes late on taking a certain COPD medication and the range in air quality limits as provided by the external computer 118 has been exceeded. This causes an alarm goes off for the client 102 to take action to fix the situation or level the environment where they find themselves. Additional alarms are sent to the external computer 118 to pass onto all authorized caregivers 146 for this client 102.

These and other examples are possible with the medical tracking application 104 and its ability to remind, encourage, use artificial intelligence (AI) and track medical events for a client 102. Basic behavioural guidance for the client 102 is established using the codified medical elements but the support of AI components can refine the original guidance and direction to a more customized level. For example, fine tuning of medication times could be invaluable for heart patients, diabetics, people suffering from chronic illnesses where pain is involved and others.

The medical tracking application 104 reports back to the external computer 118 about everything that occurs as programmed. Further, the medical tracking application 104 can use artificial intelligence to generate client inferences, such as an inference that a client 102 could be heading towards a dangerous medical event based on a myriad of possible factors. The medical tracking application 104 can also offer treatment suggestions like guidance on how much insulin is needed, how much pain medication is required based on responses to a pain scale question and others. Embodiments could also include manually confirming actions have taken place by pressing buttons on the medical computer's 108 monitor. There could be actions missed, that were performed late or that are still pending. In some embodiments, the feedback might include physical actions with scanners 132, 136, weight scales 154 and vital monitoring devices 138, 140, 142. In some embodiments, there could be fitness elements, sleep elements, environmental health and others. For example, in fitness health, the medical tracking application 104 might remind the client to perform certain activities on certain pieces of equipment, each piece of equipment having a barcode that can be scanned to confirm the activity is performed.

After receiving all these tracking activities, the external computer 118 might have one or more follow-up actions that have been programmed into the external computer's 118 memory. In some embodiments the medical feedback actions result in email messages, SMS text messages, social media messages, etc. being sent to loved ones, caregivers, medical professionals 146 and others.

In some embodiments, the medical feedback actions from the medical computer 108 result in messages being sent to other external systems 160. These systems might be integrated to the external computer 118 to support hospital EMR systems 160, private doctor systems 160, pharmacy management systems 160, etc. such that other remote web interfaces are offered to people working in those environments to see alerts and alarms from the client 102.

In some embodiments, medical analysis can be continuously performed within the medical tracking application 104, such as by an artificial intelligence (AI) component which is constantly collecting and analyzing data from all provided sources and inferred sources, allowing for fine tuning of certain medical element behaviours. For example, when a client 102 misses the optimal prescribed deadline to perform an action, such as taking a pill or performing a vital reading, this provides advanced forms of data to an AI component that can be used as input to higher-level functions of deduction. The AI component might then adjust the dosing time and frequency of reminders to add additional incentive to the client 102 if it is determined that they are having consistent issues taking their bedtime medication dose, for example. In another example, if a diabetic appears to be having issues with high glucose levels every Friday evening, it could be a pattern of letting their guard down at the end of a stressful week. An AI component that is analyzing and watching for this pattern within the medical computer 108 can adjust the amount of insulin taken by the client 102 each Friday night.

In other embodiments, the AI component might be implemented within the external computer 118. In these embodiments, it might be possible to possess a higher amount of anonymized data within the external computer 118 as it serves a wider population of clients 102. This allows for different types of large-scale decisions around populations of people, based on age, gender, time of day and other broad factors. In other embodiments, there could be AI components on both the medical computer 108 and the external computer 118. Each AI programming component might exchange messages and information to further adjust and customize medical element behaviours within the medical tracking application.

FIG. 2 shows another embodiment illustrating the main elements of the system 200. In this illustration, clients 202, 218 are looking for health tracking support and are using different types of medical computers 208, 224 to support health elements and to provide tracking support. In this embodiment, the illustrated external medical computer 222 is a cell phone sized device 222 but other devices could also be used, including, for example, a tablet computer, a laptop computer or a desktop computer.

The relationship of the support person 210 with the client 202, 218 is like a professional relationship as they are setting up and taking control of the medical tracking application 204, 220 running on the medical computer 208, 224. On the support person's 210 computer 222, they create a client record with personal and medical elements directly related to the client 202, 218. In some embodiments, the support person 210 uses a dedicated application designed and built to create and store client records and medical elements in a database at the external medical computer 222. In some embodiments, the support person 210 can also have many clients 202, 218 records created and stored on the external medical computer 222 for a range of clients 202, 218 they are supporting. Each client record is created for the client 202, 218 and corresponds to the client's profile within the external medical computer 222.

The support person 210 can then generate a client assignment identifier that corresponds to a client record in the database on the external medical computer 222. In some embodiments, the client assignment identifier is immediately generated and provided to the client 202, 218 to use at a later date. In some embodiments, the support person 210 waits until the moment they wish to assign a medical computer 208, 224 to the client 202, 218 before generating a client assignment identifier for use on that medical computer 208, 224. This would allow the support person 210 to verify the correct medical tracking application 204, 220 has been located, either by navigating to it or by downloading it from an application repository, often referred to as an app store. Once it is confirmed to be present on the client's 202, 218 medical computer 208, 224, the support person 210 can generate and enter the client assignment identifier into the medical tracking application 204, 220. In some embodiments, the client assignment identifier will be used to authenticate the client's 202, 218 identity when the medical tracking application 204, 220 sends a client assignment identifier message to the external medical computer 222.

In some embodiments, the support person 210 sets guidelines as to what capabilities are required by the client's 202, 218 medical computer 208, 224. These guidelines could include abilities like sounds, visuals, light indicators, voice control, communication abilities such as Wi-Fi, Bluetooth and others. If the client's 202, 218 medical computer 208, 224 has the necessary capabilities then the next step of assignment and download of the medical elements can take place.

In some embodiments, a support person 210 uses a direct link 206 to the client's 202 medical computer 208. In this example, a smart speaker 208 is being used. The smart speaker 208 is a highly advanced medical computer supporting capabilities like providing sound, lights and visuals. The smart speaker 208 can have one or more buttons, fingerprint readers and supports a wide range of data communications methods. The medical computer 208 supports Wi-Fi links 206, Bluetooth connections 206, RFID links 206, Internet of Things (IoT) connections 206, USB cables 206 and other communication options 206. In this example, the support person 210 would be using a direct communication 206, for example a Wi-Fi direct link, a Bluetooth link or an NFC link that is common between the medical computer 208 and the support person's medical computer 222. In this embodiment, the external support person's medical computer 222 has a direct link into the medical tracking application 204 running on the medical computer 208. The medical computer 208 has the capability of connecting 246 with supporting medical devices, including barcode scanners 232, RFID scanners 236, weight scales 234, vital reading devices 240, 242, 244, environmental sensors and other direct connections over USB.

In this example, the smart speaker 208 can be used and interactions with the client 202 can be built on audible features like voice prompts and voice commands. Reception of a client assignment identifier might happen using biometric data, voice recognition, a fingerprint or some other means. In other embodiments, a personal device like a mobile device or tablet has a Bluetooth connection to the smart speaker 208 for exchanging a client assignment identifier. The exchange or one or more client assignment identifier messages with the external computer 210 then verifies the client 202 and the encoded medical elements can be sent by the external computer 210.

Although a direct connection 206 is illustrated, the support person 210 could be using cellular protocols 214 over GSM network 216 or an IoT link 214 over cellular 216. Only after the medical tracking application 204 sends the client assignment identifier with its capabilities to their medical computer 222, can the codified medical elements be sent to the medical tracking application 204 for execution. As described in FIG. 1, the external medical computer 222 has the option of matching the capabilities to the required capabilities before sending the codified medical elements.

As described in FIG. 1, the medical tracking application 204 can then go through prompting using voice and lights to encourage the client 202 to perform a wide range of medical elements. This might include scanning medications 232, weighing medications 234, taking pulse rates 240, taking glucose readings 242, taking their blood pressure 244, etc.

In some embodiments, the smart speaker 208 can also be running advanced AI components to watch for complex correlation of events. The presence of data from actions performed by the client 202, for example vital readings, taking medication, not taking medications, poor environmental readings feeds data into the AI components. The AI subsystem might then perform notification action as supported by the smart speaker 208. Further actions can be performed to send messages back to the support person's 210 computer 222 to inform them of potential risks and dangers for the client 202 they are monitoring.

In some embodiments, the smart speaker 208 or smart wearable 224 can prompt the client 202, 218 to perform additional actions after receiving some input from another health appliance 242. For example, if a client's 202, 218 glucose reading has been received, it might indicate that a certain amount of insulin is required and can therefore suggest the correct level for the client 202, 218.

The smart speaker 208 can then use a variety of methods to report back to the external medical computer 222 regarding what has occurred. In some embodiments, the client 202 can push a button on the smart speaker 208 to indicate they have performed an action. In some embodiments, the client 202 can speak a message that gets sent back to the external medical computer 222. In some embodiments, the client 202 must perform an action confirming they have performed the necessary medical element. In these embodiments, the smart speaker 208 will continue to remind and prompt the client 202 to act until the required confirmation is performed. The action might require barcode or QR codes to be scanned on medication pill bottles and similar actions with NFC taps using NFC tags on liquid formulations, inhalers and a wide range of potential medication formulations.

The other embodiment provided in FIG. 2 shows a support person 210 supporting a client 218 who is using a smart watch 224 as their medical computer. Smart watches 224 are computer systems capable of a wide range of functions and features. Although a smart watch 224 is illustrated, the medical computer 224 could be a pendant, computer glasses or some other type of wearable medical computer. The wearable medical computer 224 must be capable of supporting a communications method that allows it to exchange messages with the external medical computer 222. Even in the case of a smart watch 224 communicating 214 through a network 216 to an external computer running on a cloud computing offering as illustrated in FIG. 1, there must be a common method of exchanging messages 206, 212, 214. In some embodiments, the external medical computer 222 uses both direct connections 206, 212 and cellular communications 214 over wide-area networks 216 to communicate codified medical elements and to receive medical tracking information. These embodiments are useful when traveling and proximity change but the need for real-time communication does not change.

The smart watch illustrated 224 is capable of downloading and running medical tracking applications 220. These advanced medical computers 224 have powerful CPUs, with highly sophisticated touchscreens, fingerprint readers, sound and vibration capabilities. These medical computers 224 offer several communication solutions including IoT (Internet of Things), Cellular, Wi-Fi, Bluetooth and NFC 212, 214. Using these connection methods, the external medical computer 222 can communicate directly 212 or use a wide-area network 216 communication method 214.

The worn medical computer 224 can also support medical reminders that require actions with external devices like barcode and QR scanners 232, pill weight scales 234, body weight scales and all kinds of vital sign monitors 240, 242, 244. In many embodiments, these vital sign monitors 240, 242, 244 for example utilize Bluetooth and low energy Bluetooth (BLE) communication methods to pair and exchange vitals information. These types of capabilities make a wearable medical computer 224 capable of supporting a wide range of client 218 needs related to tracking medical events.

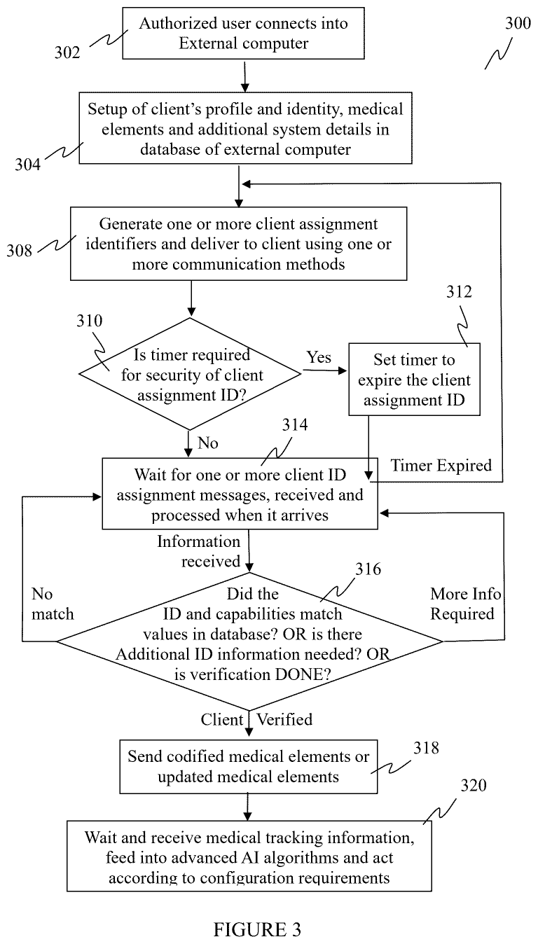

FIG. 3 shows an example dataflow 300 illustrating the main steps within the external computer to support the system. At step 302, on the external computer, an authorized user connects and begins working with the client interface provided. The authorized user might be within the same data room on a computer terminal. The authorized user could also be remote and connecting via an Internet browser using any number of computers, operating systems (OS) and browsers available to them.

At step 304, the authorized user first sets up the client's profile, identity, preferences and personal information, which can include email addresses and a cell phone number for the client that can later be used to send them reminders to additional computers they have access to. The identity can also include more personal information, allergy information, drug reactions, home addresses, doctor's name, health card numbers, social insurance number and others. The information might also include personal support contacts, including loved ones, older children, doctors, nurses, pharmacists and others who might also agree to receive notifications and alerts regarding the client's health needs and medical behaviors.

At step 304, the client's profile might also be received from another external computer system that is connected via a one-to-one integration between computer systems. In some embodiments, such integrations are usually custom built with goals for two-way interactions for the purposes of helping client's achieve better health. In some countries, there could also be a national health care system to allow integration and management of a client's health. Such system could be pharmacy based, like a pharmacy management system (PMS) or it could be a medical institution like a hospital using an Electronic Management System (EMS).

Next, the medical elements related to the client's medical profile are built up and stored in the database of the external computer 304. This information also gets saved into the remote computer's database for eventual codification before being sent to the medical computer. The medical elements can vary dramatically depending on the system, its goals and the software that has been created to meet those goals. In some embodiments, the medical elements include medication consumption needs, the types of medications, the frequency of consumption, the times of day, whether they are as needed (known as PRN) medications and how detection of consumption is determined. In some embodiments, an external system, for example the PrescribeIT system in Canada, provides some of this information for computer systems that have been integrated and approved correctly.

In some embodiments, the medical elements can include a wide range of appliance requirements, where appliances can be focused on the collection of key vital signs for the client or many other areas. For example, the collection of a glucose level might be sent to the external computer which can return a message regarding what level of insulin the client should take because of that reading. Vital sign readings could include blood pressure, heart rate, EKG, ECG, weight, blood oxygen saturation levels and other types of bodily vital sign readings. In some embodiments, the medical element might request a reading from the client's wearable monitor to determine if they have had enough activity in the day. If the client has fallen short, the medical tracking application then dynamically tells the client they must perform more walking, running, stairs or similar activities for their heart health. These and many other medical elements could be present.

In some embodiments, the medical elements include advanced guidance charts providing glucose levels and insulin requirements. A client's healthcare provider, such as, for example, an endocrinologist, primary care doctor, nurse practitioner or the like, will establish a chart of individual insulin needs based on a person's blood-sugar levels. For example, the below chart can be entered into the external computer.

| | | | | | |
|---|---|---|---|---|---|
| Blood Glucose | 70 to | 120 to | 170 to | 220 to | 270 to |
| | 120 | 170 | 220 | 270 | 300 |
| Insulin Correction | 0 Units | 1 Unit | 2 Units | 3 Units | 4 Units |

This can be customized for each client and saved in the database at the external computer. Similar charts can be created for pain relief if a client enters their pain threshold number.

In some embodiments, medical elements could also include a wide range of environmental sensors and devices. These could include temperature sensors, air quality sensors, CO monitors and even smoke detectors. In some embodiments there could be cameras available to monitor the patient and, in some embodiments, there could be requirements to show physical consumption of high-risk medications like opioids and barbiturates.

Another part of the medical elements are the required capabilities of the medical computer to support the actions required by the medical elements. In some embodiments, the authorized user will manually make these decisions by selecting menus and choices. In some embodiments, the required capabilities are automatically generated based on the medical elements selected. For example, if a medication must be taken and confirmed using an NFC tap, the medical computer must be capable of supporting NFC. In another example, if the medical element requires a reading from a blood pressure cuff, then the medical computer must support BLE (low energy Bluetooth) connections 304.

At step 308, once the database at the external computer has all the necessary personal and medical elements, one or more client assignment identifiers can be generated and delivered to the client. As discussed, the client assignment identifier can include both biometric identifiers and alpha-numeric identifiers.

In some embodiments, the authorized user might start with creating or entering an alpha-numeric client assignment identifier and then later add a biometric client assignment identifier when the client is available to provide this information physically. In some embodiments, a client might already have a state or provincial PIN associated to their identity. This identity could be for state or provincial health records, the PIN could be related to medication tracking, or it could be for other purposes. In other embodiments, a PIN might be generated for them, or the client might have selected a PIN for themselves.

Once the PIN is present, and the client is physically at a biometric reception location, they can provide their biometric. This could be at another location, for example at the pharmacy, hospital, doctor's office, safe injection site, safe consumption site, or some other monitored location. In some embodiments, the biometric is entered into the medical computer and is stored there for the highest level of security. In other embodiments, the biometric is taken at the medical computer and transferred to the external computer to be added to the database. This allows for easy swapping of medical computers should one fail or stop working correctly.

In some embodiments, once the biometric is received at a given collection site, for example a safe medication consumption site, it is given to the external computer which then propagates that information to all other related save consumption sites. It sends this biometric and the corresponding PIN to all save consumption sites to ensure a client that uses these facilities is not able to move between them and consume high-risk medications when they are not allowed, prescribed or ahead of the next scheduled dose.

In other embodiments, the authorized user generates a User Id and Password combination. This might be used with clients how are more able bodied and so entering User Id and Password is not onerous on them. Using a User Id and Password can involve entered it over again when the medical computer is rebooted, or the application is terminated and restarted.

For alpha-numeric client assignment identifiers, any number of electronic and physical methods can be used to deliver the client assignment identifier to the client 308. In some embodiments, the client assignment identifier is not human readable and would be delivered digitally and cut and pasted into the necessary medical tracking application for verification with the external computer. In other embodiments, the client assignment identifier is human readable and may comprise a username and password.

In some embodiments, the client assignment identifier could be a time sensitive username and password or a PIN code of a certain length. The use of time sensitive or elapsing access codes increases security and reduces the chance that a brute force attack could be used to guess the client assignment identifier. At step 310, a timer can be set to expire the client assignment identifier when requested by the authorized user. At step 312, a timer is set based on preprogrammed time, or using a specific time requested by the authorized user 312. If that time expires the system terminates the validity of that client assignment identifier and returned to allow another client assignment identifier to be generated. This will take place as coordinated between the client (or their authorized representative) and the authorized user. This can happen due to forgetfulness, not realizing they had a time limit or for some other reason.

At step 314, the system then waits for client assignment identifier messages related to the client assignment identifier verification process. During this waiting period a timeout may occur, when the timer has been set. Should the timer expire, the current client assignment ID has expired, and the code returns back to allow another client assignment identifier to be generated. This is then provided to the client again and the process repeats itself. In some embodiments, an authorized user must intervene to generate and provide the client assignment identifier.

In some embodiments, the client assignment identifier is pre-encrypted and provided electronically. In these embodiments, the client assignment identifier might be non-human readable characters or just a jumble of encrypted characters. In some embodiments, the client assignment identifier might be used to seed the creation of a shared symmetric encryption key. A protocol-like simple password exponential key exchange (SPEKE) complements the well known Diffie-Hellman key generation method while protecting from the man-in-the-middle.

When a message related to the client assignment identifier process is received, there could be several embodiments in the process when both a PIN or User ID and Password are used in conjunction with a biometric 316. If the setup of the client's medical elements indicates that a biometric verification is required, then an additional pass through this stage will be required. This embodiment causes the logic to return to wait for additional messages 314. This embodiment then waits for the additional information to be collecting, and in some embodiments verified.

If the information received is an actual client assignment identifier, it is compared to the values in the database of the external computer for verification 316. If no match is found, the system returns to wait for additional attempts 314. During this time of waiting the timer could expire which causes the need to communicate to the client and regenerate a new client assignment identifier. In many embodiments there could be keyboard typing errors and re-entering of the client assignment identifier. If there is a configured number of maximum attempts, whether it is per hour, per day or forever, then it is possible the account could get locked out 314.

In some embodiments, the client assignment identifier message contains the capabilities of the medical computer. When present, a further check is performed to verify whether any received capabilities match the corresponding required capabilities of the medical computer. If the capabilities are lacking, a message could be sent to the medical computer to inform the client. The system will then return to wait for another client assignment identifier 314, for example, from a different and more capable medical computer.

In those embodiments where additional client verification is required, another processing step is required either within the medical computer or within the external computer 316. In some embodiment this additional information is the client's biometric information, used for personal and advanced verification that the client is the person they say they are. If the biometric has not been provided to the external computer yet, or if the medical computer has not provided confirmation that full verification is complete, the system returns to wait for that required information 314.

In some embodiments, it is possible the medical computer stores either the alpha-numeric client assignment identifier, stores the biometric client assignment identifier or saves both types of client assignment identifiers. If the medical computer indicates in the client assignment identifier message that full verification of a client is complete 316, it provides a shared identifying part of the client. For example, this could be the person's full name, it could be their health card number, drivers license or some other identity of the client. Once received and the client record is found, the external computer sends the codified medical elements or updates to the medical elements 318.

In other embodiments, the external computer provides a stored biometric to the medical computer and requests a biometric input that must match the provided input. In this embodiment the external computer returns to wait for additional confirmation that the biometric input matches the biometric just provided to the medical computer 314.

In other embodiments, the external computer confirms the accuracy of the alpha-numeric client assignment identifier but then requests a biometric for the client be collected and returned to the external computer 316. This causes the logic to flow back up to step 314 where the additional biometric information is collected 314 and returned for verification 316. In some embodiments, once this is done the first time the medical computer might have the choice to store the biometric in a secure local memory to save the time and costs of transmitting the biometric repeatedly.

If the information received from the medical computer indicates that the client has been fully verified, or if the external computer has performed all the necessary verification, the external computer sends the codified medical elements to the medical computer to execute 318.

In some embodiments, the external computer then sends the codified medical elements to the medical tracking application 318. In other embodiments, the external computer might send codified updates to the medical elements 318, as it knows the medical tracking application has stored the last full codified medical elements. Sending just updates is a faster, more efficient and cost-effective way to continuously keep the medical tracking application up to date 318. In the final client assignment identifier message, the medical tracking application might even provide the date or version number of the last medical elements it received. This would then be used to guide the external computer as to what should be sent or resent to the medical tracking application for that client 318.

The external computer waits and receives medical tracking information from the medical tracking application and performs any alert notifications as configured by the authorized user for this client 320. This could lead to messages and actions to configured professionals, caregivers, PSWs and other health care professionals to alert them to events happening with the client at the medical computer. The medical tracking information can also be fed into advanced AI components where a larger amount of computer power can be applied to look for issues, dangerous situations, potential problems and dangers for the client. This then results in messages being sent to the client on the medical computer or to all approved professionals, caregivers and others.

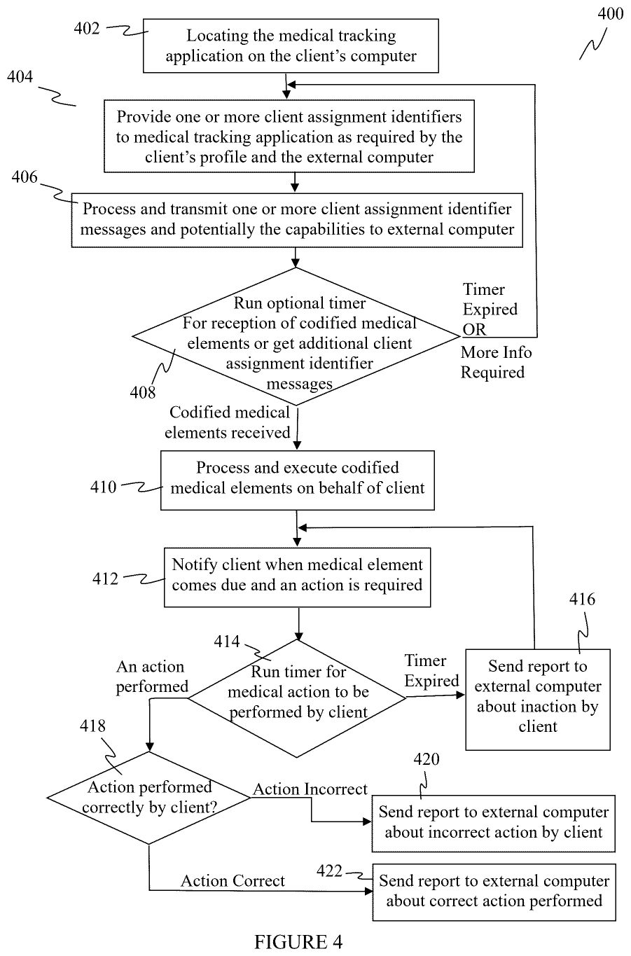

FIG. 4 shows an example dataflow 400 illustrating the main steps within the medical computer to support the system. At step 402, the medical tracking application is located on the medical computer. In some embodiments, the process of locating involves navigation through the user interface to find and launch the medical tracking application. In embodiments where cell phones, tablets or laptops are used, there are often hundreds of applications loaded and pre-loaded onto a medical computer. In other embodiments, a specialized application must be downloaded from a specific location onto the medical computer. This process is commonly done from application stores where applications can be purchased or downloaded for free to support such an activity as medical tracking. In other embodiments the medical tracking application is running on a dedicated medical computer that has no other purpose then to run the medical tracking application.

At step 404, once the medical tracking application is running, the steps to identify the client can commence. One or more client assignment identifier can be exchanged based on the design of the system and its current state. In some embodiments when an alpha-numeric client assignment identifier is used, the client or the client's representative can cut and paste the client assignment identifier from an email, social media message, a private and secure medical email solution, or some local messaging system into the medical tracking application. In other embodiments, the client assignment identifier might be read off a printed piece of paper, read off a web page, or read off a cell phone screen and typed manually into the medical tracking application. For example, the client assignment identifier could be a username and password, a one-type passcode that expires, or some other form of password.

In some embodiments, after the very first time the client assignment identifiers have been provided by the external server in a client assignment identifier message, the medical tracking application stores them in a secure memory, for example a secure conclave memory at the medical computer. This would be the most efficient way to save time and speed when getting the client up and working. In some these embodiments the medical tracking application first verifies the one or more locally stored client assignment identifiers 406 and re-loads the previously received medical elements 410. It sends off a client assignment identifier message with an indication that the client has been verified and provides some internally agreed upon name or identification for who that client is. When this message goes to the external computer it might respond by providing any updated codified medical elements that have changed since the last codified medical elements were sent to the medication tracking application.

In other embodiments, the client might immediately request a biometric of some kind from the client, such as, for example, a facial scan, fingerprint, retinal image, deep palm scan and others 406. The medical tracking application might then compare the biometric to all biometrics stored in the medical computer looking for a match. In some embodiments, if no match is found, the medical tracking application could send the biometric received in a client assignment identifier message to the external computer to see if a match exists there. If a match is found, the external computer sends a message confirming the biometric belongs to a client named <First Name> <Last Name> accompanied by the codified medical elements 408.

In another embodiment, the medical tracking application asks for a biometric first, then takes an alpha-numeric, like a PIN or a User ID and Password. In this embodiment the medical tracking application then has the choice to transmit just the alpha-numeric to the external computer, which would be faster and more cost effective than sending a biometric 406. Once the client assignment identifier is confirmed by the external computer, the biometric can be confirmed and permanently stored so that only the biometric is required 408 in the future.

In another embodiment only an alpha-numeric client assignment identifier is requested by the medical tracking application. This is then transmitted to the external computer in a client assignment identifier message, and the external computer may in turn request additional information 408. If the external computer can confirm the alpha-numeric client assignment identifier, it might then realize that a biometric is also required. The logic then flows from that request 408 back up to requesting another piece of information from the client to confirm who they are 404.

At step 406, once the medical tracking application receives one or more client assignment identifiers based on the embodiment, the medical tracking application processes the client assignment identifier. This process might involve checking for the correct length, performing an internal comparison to previously stored client assignment identifiers, performing some amount of verification, formatting and syntax-checks of the values provided or other potential local verifications. This then generates a client assignment identifier message, including the one or more client assignment identifiers or indicating verification is complete, depending on the embodiment.

In some embodiments, the medical tracking application also collects a list of capabilities and attaches them to the message containing the client assignment identifier. In some embodiments, a biometric could be received and confirmed. In some embodiments, a biometric is processed and prepared for transmission to the external computer. This message will eventually be sent to the external computer so it can verify the client assignment identifier and ensure the capabilities of the medical computer are equal to or greater than the required capabilities to execute the medical elements 406.

In some embodiments, various forms of encryption are used between the external computer and the medical tracking application. The channel between them might be secured using a protocol like transport layer security (TLS) and additional security over shorter links like Wi-Fi Protection Access (WPA3) and a combination of these methods might be employed. In some embodiments, all messages that are exchanged between the external computer and the medical tracking application are encrypted. This helps to ensure that no matter what path the messages take, they are secured end-to-end, even if the connection could be insecure.

In other embodiments, the client assignment identifier has been pre-encrypted and provided electronically to the medical tracking application in a client assignment identifier message. In these embodiments, the client assignment identifier might be non-human readable characters or just a jumble of encrypted characters. The client might cut and paste a string of characters into the necessary field for the medical tracking application to process 406. In other embodiments, the alpha-numeric component of the client assignment identifier might be used to seed the creation of a shared encryption key. A protocol like simple password exponential key exchange (SPEKE) complements the well known Diffie-Hellman key generation method while protecting from the man-in-the-middle.

At step 408, once the client assignment identifier is sent, the medical tracking application runs a timer to wait on the reception of a response from the external computer. The response could be the codified medical elements, or it might be a request for additional client assignment identifier message exchange, for example “please acquire biometric of this <TYPE>”.

If the timer expires, the medical tracking application may re-acquire the client assignment identifier from step 404 and resubmit it to the external computer. In some embodiments, the person entering in the client assignment identifier may mistype a character, making it invalid and therefore ignored. In some embodiments, the external computer might respond with a follow-on message saying that the client assignment identifier was not recognized. In embodiments where higher security is required, the failure to enter the correct key may result in the generation of an incorrect encryption key and the received message becomes undecipherable. In some embodiments, the external computer might only allow a certain number of failures per hour, per day or over a week.

At step 410, if the client assignment identifier is correct and the capabilities are sufficient to meet the required capabilities, the external computer will send the codified medical elements. These are then processed and executed on behalf of the client to guide the operation of the medical tracking application.

At step 412, execution proceeds to notify the client when various medical elements are due, and an action is required.

Each time a medical element comes due, at step 414, a timer will be run to ensure the action is performed by the client in a timely fashion. In some embodiments, the time allotted for the medical element is clearly indicated. For example, there is a medication to be consumed, and after 60 minutes, it is considered late. However, if that same medication is not taken after 3 hours, it is considered missed. These time limits can be provided through the codified medical elements as configured by the authorized personnel at the external computer.

If the timer does expire, at step 416, a report is sent to the external computer about the client's inaction related to a medical element. With this report, the external computer can then generate alerts in the form of email messages, SMS text messages and others to warn caregivers that something negative has occurred. This is part of the configuration performed by the authorized personnel at the external computer.

If an action is performed at step 414, then at step 418, a second test is performed to see if the action was performed correctly. If the action was performed incorrectly, then at step 420, a report is sent to the external computer about the client incorrect action performed. In some embodiments, for example, the client might have to take a medication but grabbed the wrong bottle and took the pills that were inside. In another example, the client was supposed to take their blood pressure but took their heart rate instead. If the action performed was correct at step 418, then at step 422, a report is also sent to the external computer to report the correct action taken by the client. In some embodiments, even correct actions will generate an email message, a SMS text message, or a message to EMR hospital platforms to inform professionals of the action performed.

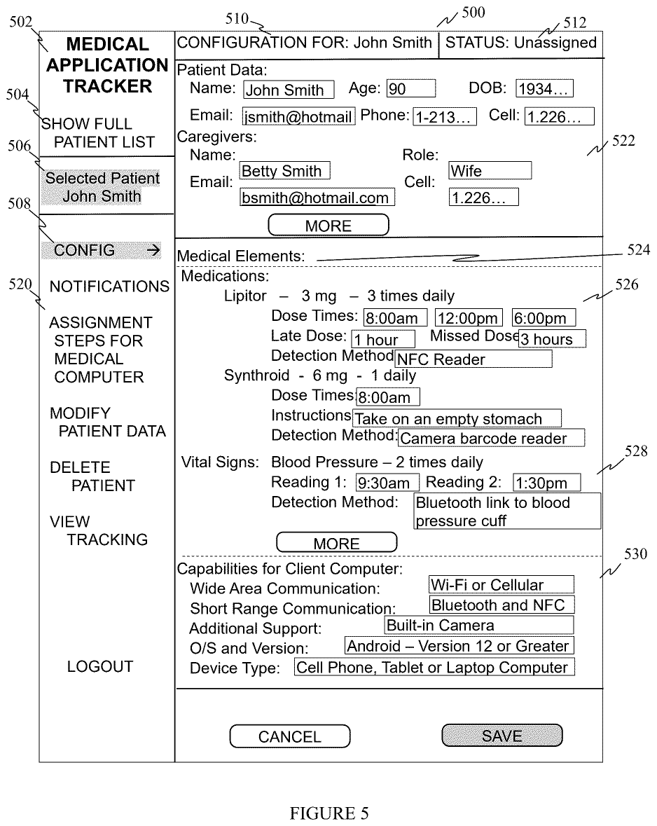

FIG. 5 shows an illustration of one example configuration screen 500 for a client 506 at an external computer. This Medical Application Tracker 502 at the external computer represents one example embodiment for a multitude of potentially differently structured user interfaces (UIs). UIs can be built using advanced screens, graphics, options, picklists, hamburger menus and choices for an authorized user. This simple embodiment UI 500 is built using static squares and circles to simply the illustration for presentation and is not intended to represent the only way to build a screen designed to configure clients on a medical tracking application.

Shown on the left panel 502 is a title but also several selectable menu options. The authorized user can jump to a new screen to see the entire full patient list 504. This screen would be where the authorized user jumps from patient to patient or creates a new patient. When a patient is created or selected on this screen, the authorized user first arrives to the patient's configuration screen 508. There are other options 520 for configuration notifications, including assigning the patient to a computer, modifying the patient's data, and deleting the patient and viewing tracking information for the patient. There could be other potential menu options for a given patient, for example, unassigning them from a computer. In some embodiments, the “Assign to Computer” option will toggle to “Unassign from Computer” after the assignment is successful.

The top line of the screen shows a title for the screen currently active. In this example UI 500, it would be the ‘Configuration For: John Smith’ 510. It also shows John Smith's current assignment status, which is ‘Unassigned’ 512. Below that title is the current information provided for this patient 522. In FIG. 5, the patient data includes a modest list of personal information, including, for example, their age and date of birth (DOB) and contact information. Including an email address and cell phone number allows the external computer to use these in other parts of the system to send email and text alert messages and even send the client assignment identifier if desired.

The patient data section can also include caregiver information. In FIG. 5, the name of the caregiver and the caregiver's relationship to the patient are the main personal details. This is followed by contact information, which is an email address and a cell phone number. Including an email address and a cell phone number will allow the external computer to use these values later for alert messages. In some embodiments, every email address and cell phone number are verified with a follow-on onboarding message to the patient and caregivers to ensure the information is accurate and they accept the responsibility of monitoring alert messages. In some embodiments, there would be more information, as indicated by the MORE button, but in FIG. 5, not all data has been shown for ease of presentation. For example, the patient might have 5 or 6 more caregivers, but their information would be similar to what has already been presented.

Following the Patient Data section is the Medical Elements section 524. The medical elements 524 can be made up of medication elements 526 and vital signs elements 528. In some embodiments, there could be fitness elements, sleep elements, environmental health and others. For example, in fitness health, the medical tracking application might remind the client to perform certain activities on specific equipment, each piece of equipment having a barcode that can be scanned to confirm the activity is performed. For FIG. 5, the medications 526 and vital signs 528 are the two medical elements to be configured, monitored, reminded and tracked.

In FIG. 5, the medication elements start with the drug Lipitor, which the patient takes 3 milligrams per dose, 3 times daily. The dose times are provided as 8:00 am, 12:00 pm and 6:00 pm in this example. There are configuration settings for what is considered a late time and a missed time for taking the medication. Also included is the detection method that must be used when confirming the medication has been consumed. In this example, an NFC reader will be used to detect an NFC tag on the pill bottle. This will later be used to help determine the required capabilities of the medical computer.

The next medication shown in FIG. 5 is Synthroid and it is taken in a 6-milligram dose just once per day. The dose time is 8:00 am and patient instructions have been provided to ensure the medication is taken on an empty stomach. The detection method in this embodiment is a camera-based barcode reader. This detection method would employ an onboard (built-in) computer camera to detect barcodes, QR codes, Q-codes and all other forms of coded values. This setting would also affect the required capabilities of the medical computer.

The second part of the medical elements in FIG. 5 is for tracking vital signs 528. The authorized user has requested the patient to take their blood pressure twice per day at 9:30 am and 1:30 pm. These are specific times but in other embodiments, the times might be directly related to when a medication like Lipitor is taken. In these embodiments, once the user scans the Lipitor the configuration would say take your blood pressure 1.5 hours after taking your dose of Lipitor. The detection method for confirming the blood pressure, and receiving the results, is a Bluetooth link to a blood pressure cuff. In some embodiments, the patient will have to purchase a specific blood pressure cuff, or one might be provided for them to ensure accuracy and quality of the readings. Further correlation of the timers and their specific time could be affected by AI components running on the client's computer as shown in FIG. 7.

The last section shown in UI 500 is the Capabilities section 530. There are many embodiments that could be displayed in this section, and only a subset of all the potential capabilities is presented. The areas displayed include wide area communications, short range communications, additional support, operating system (O/S) and OS version information and device type. In some embodiments, the external computer might auto-fill these values and allow the authorized user to override or modify some of them. In other embodiments, the authorized personnel might select all the capabilities needed. In some embodiments, the authorized user might ignore the capabilities section and leave it for the patient to pick a computer that is suitable to them. Once the desired information has been selected and inputted, the authorized user can then CANCEL the changes they've made, or they can SAVE the changes to include in the patient's configuration.

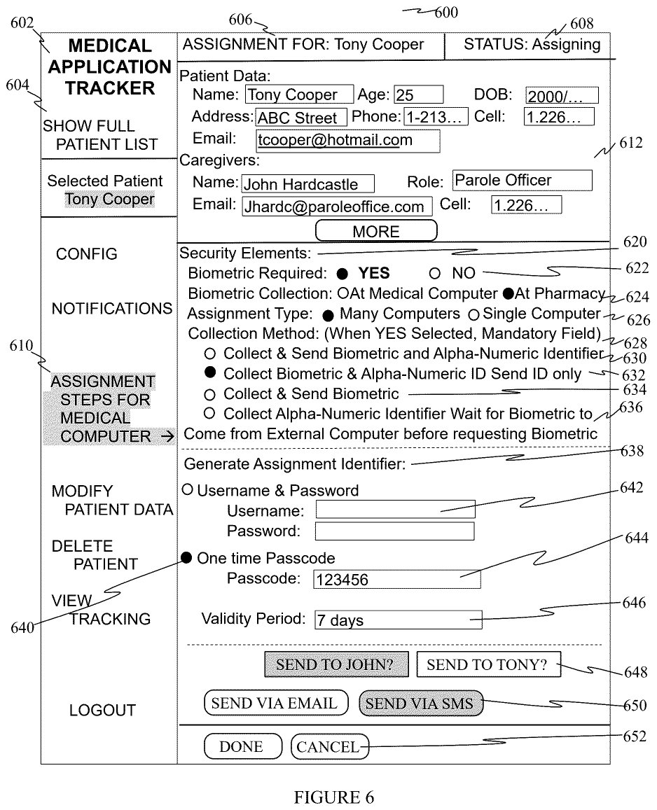

FIG. 6 is an illustration of one example assignment screen UI 600 at an external computer for assigning a medical tracking application to a client. This is just one embodiment of a UI for generating and selecting a client assignment identifier and does not use dynamic graphics, options, picklists, hamburger menus and choices for an authorized user. This simple embodiment UI 600 is built using static squares and circles to simply the illustration for presentation and is not intended to represent the only way to build a screen designed to assign a medical tracking application to a client.

Similar to FIG. 5, the title 602 is presented with various options 604 that can be performed by the authorized user. In this example screen UI 600, the authorized user has selected Assignment Steps For Medical Computer 610, meaning the currently selected patient is ready to be assigned to a computer. The top line indicates we are performing an assignment for Tony Cooper 606 and the status indicator indicates we are in ‘Assigning’ mode 608. Tony Cooper seems very young, and his main caregiver is a Parole officer so it appears that Tony might be dealing with substance abuse and is on a witnessed dosing program offered by the government. The patient information is displayed 612 for convenience of the authorized user, and it will provide some background on the user during the assignment. In some embodiments, the assignment screens might be modal screens and overlay whatever might have been previously on the screen before performing the assignment step.

FIG. 6 shows the Security Elements section 620 presents one embodiment for the many previous descriptions of how security could work in the system. This one example is shown for convenience, and no way diminishes the multitude of previous embodiments described in the patent.

In the first step the authorized user determines if a biometric is required when accessing the medical computer 622. This example shows that a biometric is required to operate the medical tracking application. Next the biometric collection method is selected between the medical computer or within the pharmacy 624. In this example the pharmacy is the selected biometric collection location. Pharmacies supporting the solution would be issued a biometric collection station that connects to the external computer to provide the biometric. There are many pharmacies that support witnessed dosing, and it is one of the many places discussed earlier where a client's biometric could be collected to be used by the external server.

Next, the authorized user selects the assignment type for the client, which could be at many computers or at a single computer 626. Selecting many computers as shown indicates that the collected biometric might have to be shared with many medical computers in the system or the biometric received at the medical computer must be returned to the external computer for verification. Both these embodiments have been discussed in detail.

Next, the collection and use method that is shown as mandatory when the biometric required parameter is set to YES 628. This parameter allows different exchanges of client assignment identification messages between the external computer and the medical computer. The first option allows the medical computer to collect both the biometric and the alpha-numeric identifier and send them both to the external computer for verification of the client 630.

The second option requires the collection of the biometric and the alpha-numeric identifier but only the identifier is sent to the external computer for verification 632. This allows for several embodiments, for example in one embodiment the biometric has already been sent by the external computer to the medical computer and has been saved in a secure memory. This allows the external computer to respond with a client assignment identification message indicating the alpha-numeric identifier is valid and also provide the correct matching biometric. In one embodiment, this procedure is followed every time. In another embodiment, the external computer only sends the biometric the very first time as it knows the medical tracking application will save that biometric for that client in a secure memory within the medical computer 632. This is a very flexible choice, and the authorized user has selected this choice from the pick list.

The next choice requires the medical computer to collect and send the biometric to the external computer every time the client wants access to the medical computer 634. The final choice is to require the medical computer to collect an alpha-numeric identifier and wait for biometric to come from the external computer before requesting the client's biometric at the medical computer 636. This option might be used for higher security where the biometric is not stored and can be updated at the external computer more easily. For example, perhaps the client needs to change biometric as they broke a finger or the existing biometric is poor quality and a new one is required 636.

FIG. 6 shows the generate assignment identifier section 638, providing an example of several embodiments for creating and sharing the client assignment identifier. For convenience, in this example, many different embodiments are illustrated at one time. In some embodiments, the system would be designed to work in only one specific way. By selecting between using a Username and Password 642 or using a One-time Passcode 644, the authorized user has two different ways to authorize the medical tracking application. This is a radio button, so only one button can be selected at a time 640.

In this example, the authorized user has selected 640 to use the one-time passcode method of security 644. In some embodiments, this selection would require the use of a validity period for that one-time passcode 646. In the UI 600, the validity period 646 can be applied to either the username/password 642 or the one-time passcode 644. In this example the passcode will be valid for 7 days and then the client will have to request a new one. If this client is dealing with substance abuse perhaps one every 7 days, they must check in with a pharmacist to prove they have been taking their methadone or similar treatments faithfully.

FIG. 6 provides additional embodiments by allowing the passcode to be sent to various individuals 648. In this embodiment, the passcode can be sent to either the patient or one or more of their caregivers 648. In some embodiments, only the patient might be allowed to receive the passcode 648. However, when dealing with very elderly patients or patients with addiction issues, it can be valuable to have a trusted caregiver deal with the complicated steps of setting up or monitoring the client's passcode and, in come cases, tracking all behaviours and interactions with the medical tracking application. In the example shown in FIG. 6, John is receiving the new passcode and will provide this to the person on parole that they are responsible for.

The final choice shown in the UI 600 allows the client assignment identifier to be sent via email or via SMS text message 650. In this embodiment, the client assignment identifier could even be sent to both people to ensure it is received and acted upon before the validity period expires. Once all the selections are made, the authorized user will indicate they are DONE, or they can CANCEL 652 what they have just configured. When DONE is selected, the client assignment identifier will be issued and sent following the instructions provided.

FIG. 7 shows an illustration 700 of another embodiment where the main elements of the system are illustrated. There are two embodiments represented in the illustration, but this does not restrict there being many other similar embodiments with adjustments to small elements of the message exchange, authentication steps and verification process.

The first embodiment shows a first client 702 holding a medical computer 714, where the first client 702 is standing in front of a large pharmaceutical dispensing machine 706 that houses medication 708 in a secure fashion. In this embodiment, the medical computer 714 could be a personal cell phone, tablet computer, wearable device or some other type of small mobile computing device 714. The pharmaceutical medication machine 706 most likely houses drugs that are high-risk, additive and probably a controlled substance 708.

The second client 704 is about to place their hand on a medical computer 716 that is connected 718 to the pharmaceutical medication machine 706. This small medical computer 716 accepts a biometric for personal identification. In some embodiments, there could also be different types of keyboards, such as, for example, a QWERTY keyboard or just a numeric keypad for inputting alpha-numeric client assignment identifiers.

The first step is for a professional 734 or an authorized user 740 to set up a client record in the database 752 of the external computer 754. The client record then corresponds to each client 702, 704 and would define what is possible in the system 700 and what medical elements suit their particular situation. If for example the pharmaceutical machine 706 was designed for opioid additional treatments, it could be dispensing methadone, suboxone, naloxone or a similar medication used to treat opioid addiction in adults 702, 704. When such a serious treatment is required, a professional like a doctor 734 or an authorized user such as a pharmacist 740 is well suited to determine the frequency of treatment, method of access and then to follow up and monitor the client's 702, 704 progress.

Various communication means are supported within the system 700 to facilitate the movement of data and interactions. One or more networks 720 are used for wide-area communications. These networks can interconnect using various gateway methods and standard protocols. Cellular wireless networks 720 offer seamless interconnectivity with world-wide networks like the Internet 720. Connections into these networks can vary from direct connect fibre connections 724 to radio frequency cellular connections 722. The types of these cellular networks 722 in the GSM, 5G, UMTS category have been discussed in earlier slides. Other types of networks 720 can include private virtual networks (PVN) and public networks like the Internet.

An authorized user 734 can use various means to connect 724 using personal computers 736, desktop computers 736 and software like Internet browsers to communication and interact with the external computer 750. The external computer 750 can be a cloud computer service 750, a back-office 760 private computer server, a smaller desktop server 750 and many other choices. It provides communication and CPU services 754 that allow access to a database 752 for storing any number of client records. An authorized user 734, 740 connects with the external computer 750 to then create a client record in the database 752. In some cases, the external computer could be running within a special facility like a pharmacy 742. In this embodiment, the localized external computer 742 would have to have a connection 724 and relationship with the medical computer 706. It might be feasible for a large pharmacy chain 760 to offer such a collection of external medication dispensing machines 706 all connected to their internal back-office computers.

In this embodiment, the client record indicates that a biometric is required to access the medical tracking application running on the medical computer 714, 716. There are several embodiments discussed to allow this to happen. Depending on the circumstance and the client 702, 704, the client 702, 704 may be asked to go into a specialized location to provide a biometric. This could be a fingerprint, a palm print, retinal scan, facial scan, a subdermal implant scan, or some other type of person biometric. Circumstances can vary in this illustration 700 as clients 702, 704 are in different stages of recovery and confidence. As trust increases for a given client 702, they might be given more independence and freedom. If a client 704 is in the early stages of recovery, they may be given less independence and freedom.

In one embodiment, the client 702 might have had their client record created and are quickly able to use the medical computer 706 using their personal computer 714. This might be a mobile computer, it could be a tablet, laptop or wearable computer 714. It could communicate using a USB connector, for a high degree of safety or it could use a short-range communication method 712 like encrypted Bluetooth, RFID or near field RFID also termed NFC. The medical computer 706 could be equipped with both Bluetooth and NFC compatible hardware 710 to allow for many ways of dispensing the medication 708 held within.

In this embodiment, the client's computer 714 is running the medical tracking application and is acting as the medical computer 714. In some embodiments, the client 702 would enter their PIN or User ID and password to give to the external computer 750 for verification. Then the external computer 750 would verify that and tell the medical tracking application to request a biometric confirmation from the client 702. In another embodiment, the client 702 simply has to enter their biometric confirmation into the client's own computer 714 and the medical tracking application running on the client's computer sends a verification complete indication in the client assignment identifier message. Once verified, the external computer 750 could send back the codified medical elements or codified updates to the medical elements with an access code needed by the dispensing equipment 706. The client 702 would then hold up their phone to the NFC tap area 710 and the code would be given to the dispensing equipment 706 for verification. Once verified and activated by the necessary code, the dispensing equipment 706 dispenses the medication 708 amount indicated by the code provided. There could be a range of code values that would correspond to dispensing different types, numbers and volumes of pills based on the client's 702 configured needs.

In another embodiment, the medication dispensing machine 706 has a communications link 722 with the external computer 750 and is able to exchange a full range of secure messages 728 with the external computer 750. In this embodiment, the client's 702 personal computer 714 might only be used for biometric input in order to launch a simple biometric confirmation application that performs a short-range communication 712 with the medication dispensing machine 706. The communication 712 might be USB, Bluetooth, RFID or NFC to exchange a confirmation and the identity of the client 702 who is requesting medications. In this embodiment, the medication dispensing machine 706 is the medical computer and it communicates with the external computer 750, indicating that verification is complete with the identity of the client 704 trying to use the medical computer 706. The client assignment identification message provides all this information and the external computer 750 replies with the codified medical elements to guide the release or non-release of medication 708. Should the client 702 be attempting to take medications 708 at an inappropriate time, such as, for example, too soon, the medical computer 706 could display a message with the correct time.

There is also an embodiment where the application running on the client's 702 computer is running a scheduler and informs the client 702 when it is time to request medications 708. This can come from the external computer 750 using a message passing protocol 726 or from the medical computer 706 directly when the client 702 tries to perform their first extraction of medications 708.

In another embodiment, the client 702 has previously provided their fingerprint at a secure location 760 while an authorized user 740 oversees that process. The secure location could be a medical facility, a pharmacy, a safe dispensing site, a hospital or many other locations. The client 702 may have provided their fingerprint at a specialized biometric reception computer 744 or they might have done it with their own computing device 714, running the medical tracking application, in front of an authorized personnel 740. This embodiment requires less trust of the client 702 as their biometric can not be inappropriately used by another person trying to acquire the drugs 708 inside the medication dispensing machine 706. Once collected by the biometric reception computer 744, the biometric can be sent to the external computer 750 and stored in the database 752. Once saved in the database 752, the biometric can then be propagated as required. Should the client 702 need to use the attached biometric reader 716, the biometric can be compared by the external computer 750 for final verification.

In another embodiment, a client 704 must go into an authorized location 760 and be monitored by an authorized person 740 to provide a biometric to a biometric collection computer 744. This client 704 might have more serious drug issues and cannot be trusted to perform as expected. Once the biometric is collected by the biometric collection machine 744, it can be transferred to the external computer 750 to propagate to all medication dispensing machines 706 that the client 704 might potentially visit. In some embodiments, the biometric might be sent to a specific medication dispensing machine 706 when the client 704 actually turns up there. In this scenario, the medication dispensing machine 706 is the medical computer 706 and is running the medical tracking application. The medical computer 706 has a biometric collection reader 716 that is connected 718 in some manner. This connection could be a USB connection, it could be hardwired or built into the medical computer 706, it could use a wireless link like Bluetooth, RFID or NFC or other advanced connection methods.

Once the client 704 is verified using one or more exchanges of client assignment identification messages, the medical computer 706 receives the codified medical elements corresponding to the client 704. In this scenario where the client 704 might have a more serious problem, the medical elements might require external support to prove they have taken their medication 708. In this example, the medical computer 706 has a mounted camera 732 which can take pictures or video as directed by the medical computer 706. In other embodiments, there could be a blood pressure cuff, EKG, pulse rate indicator or some other device to be used to verify the client 704 has done what they are supposed to. In this example, the medical computer 706 might prompt the client 704 to place the medication 708 on their tongue and press a button for the camera 732 to take a photograph of them. This photograph would then be uploaded to the external computer 750 so that at any time, authorized users 734, 740 can confirm the client 704 is performing as they should. If they are not performing, their access to the medical computer 706 might be revoked and they will be forced to return to daily visits to the pharmacy 760 where full witnessed dosing is required.

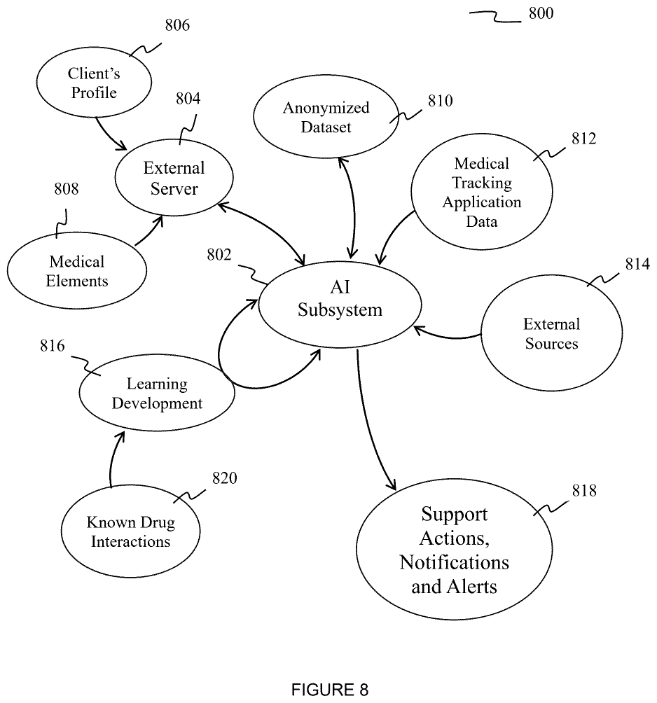

FIG. 8 shows an illustration 800 of an embodiment of an AI subsystem 802 with one or more AI models that could be used in the medical tracking application to augment traditional software logic and operation procedure. The AI subsystem 802 is based on processing of multiple disjointed inputs to correlate the data with advanced learning models to improve medical outcomes and support alerting and notification procedures 818.

The AI subsystem 802 relies heavily on a supervised learning model, where the datasets are categorized across multiple classifications. In some embodiments, the categorization focuses on the client's characteristics found within the client's profile 806, such as, for example, age, gender, race. These characteristics can be anonymized and then cross referenced to the activities being performed within the medical tracking application based on the medical elements 808. Using a limited memory AI learning pattern, the AI subsystem 802 uses the actions performed and not performed by the client, as reported by the medical tracking application 812, to make predictions regarding the client's future behaviour.

Initially the AI subsystem 802 receives data from the external server 804. For example, the data may be primarily composed of the client's profile 806 and medical elements 808. Medical elements 808 can include medications, vital sign configuration, operation data, external medical devices and environmental sensors that are expected to be connected.

The AI subsystem 802 can use data in the client's profile 806 as a starting point to place or classify the client into various categories that can affect the approach to health and maintenance of their health. A person's age and gender, for example, are example indicators of patterns that can guide advanced learning development 816. In some embodiments, the AI subsystem 802 is running within the external server 804. The AI subsystem 802 is then provided with large amounts of anonymized client profile data 806. As new client profiles 806 are added, the external server 804 scrubs the data removing personal details, such as, name, address, phone numbers, etc., such that the data becomes anonymized data 810 before providing the anonymized data 810 to the AI subsystem 802. This anonymized data 810 grows as the number of clients in the external server 804 grows. In some embodiments, the AI subsystem 802 is operating in its own environment, such as, for example, a unique cloud computing environment, and can communicate with multiple external servers 804 where the anonymized data 810 being received can grow exponentially.

Once deployed, the medical tracking application feeds data 812 to the AI subsystem 802 from a variety of sources. Medical tracking application data 812 forms the most immediate and real-time bases for data and building predictive behaviour models. This provides actual actions performed by the client, timers that have expired when actions are not performed, and actions that are performed incorrectly, such as, for example, taking the wrong medication when instructed to take a medication. Each of these inputs are accompanied with time of day. Time of day is important input information to help correlate the anonymized data to the client's age and gender to understand potential outcomes. For example, what is the likelihood that an elderly male client will forget their medications at 9:00 pm, just before bed? Therefore, the AI subsystem 802 should send out alarms early to caregivers and loved ones at 9:15 pm when the client fails to take their medications on time at 9:00 pm.

Additional inputs from external sources 814 can lead to additional learning development 816. Readings from vital sign devices and environmental sensors can be early warning signs that the patient needs attention. For example, if a glucose reading from a glucose monitor falls outside an acceptable range, the patient is over 75 and has additional commodities like COPD or heart disease, then immediate warnings and alerts should be sent to the client's computer as well as to the external server.

Learning development 816 can also use known drug interactions database to input additional actions into the AI learning process. Taking known drug interactions 820 into account, the AI subsystem can watch for medication consumption that falls out of acceptable gap and dosing parameters. For example, if a patient's thyroid medication must be taken at least 2 hours from their heart medication, and the patient takes their thyroid medication 1 hour late before proceeding to take their heart medication at the normal time, the AI subsystem can use its collected information to forewarn the patient to postpone taking their heart medication for an additional hour to be safe from potential drug interactions.

These and many other complex learning processes can be built up with the AI subsystem 802 over time and with a growing database of anonymized data 810. As shown, the AI subsystem 802 and external server 804 can feed anonymized data back into the anonymized dataset 810 to grow the amount and quality of the data. AI components could also be working within the external server 804 to correlate and improve the anonymized data 810 so that requests from the AI subsystem 802 for that data results in more accurate and better-suited data for the client age, gender and time-of-day predictive patterns.

1. A method for generating notifications or alerts based on medical activities relating to codified medical elements from a medical tracking application and assigning the medical tracking application to a client using an external computer, the method comprising:
creating a client record with personal and medical elements and storing the client record in a database at an external computer, the client record corresponding to a client;
storing one or more client assignment identifiers in one or more databases, the one or more client assignment identifiers corresponding to the client to allow for authentication of a client identity using a medical tracking application running on a medical computer and the external computer, the client identity corresponding to the client;
exchanging one or more client assignment identifier messages between the medical tracking application running on the medical computer and the external computer, wherein the one or more client assignment identifiers comprise or indicate capabilities of the medical computer allowing the external computer to determine whether the capabilities of the medical computer support requirements of the medical elements;
upon the client using the medical tracking application, authenticating the client by processing the one or more client assignment identifier messages to verify the client identity using one or more database lookups of the one or more databases;
matching the capabilities of the medical computer against the requirements of the medical elements to further authenticate the capabilities of medical computer to support the medical tracking application to execute the codified medical elements;
codifying, at the external computer, the medical elements from the client record for the client, wherein the codified medical elements comprise one or more required actions to fulfil the medical elements, the one or more required actions comprising at least one of biometric collection, barcode scanning, QR-code scanning, RFID communication and weight scale readings;
sending the codified medical elements to the medical tracking application running on the medical computer for execution by the medical tracking application, the medical tracking application executing the codified medical elements to: notify the client when one or more medical elements have come due and at least one encoded action is required; monitor the client performing the at least one required encoded action using data received from the one or more connected medical devices; and determine whether each action Performed by the client was Performed correctly or incorrectly;
receiving, at the external computer, a plurality of medical activities relating to the codified medical elements from the medical tracking application running on the medical computer; and
generating a report and alerts for authorized users based on the plurality of medical activities relating to the codified medical elements, wherein the report comprises correct actions, incorrect actions, and inactions of the client related to the codified medical elements.

2. The method of claim 1 wherein the one or more client assignment identifiers are securely stored in the medical computer and used for verification after a first verification has been successfully completed.

3. The method of claim 1 wherein generating of one or more of the client assignment identifiers is implemented at the external computer, uploaded to the external computer or implemented within the medical computer.

4. The method of claim 1 wherein the sending the codified medical elements comprises sending only changes that have been made since the codified medical elements had been sent to the medical tracking application running on the medical computer.

5. The method of claim 1 wherein the medical computer running the medical tracking application is one of a personal mobile computer that is property of the client and a shared computer across a plurality of clients, wherein the plurality of clients includes the client.

6. The method of claim 1 wherein each of the one or more client assignment identifiers is one of multiple values composed of one or more alpha-numeric values, one or more biometric signatures or a combination of both.

7. The method of claim 1 further comprising: relaying messages, using the external computer as a proxy agent, between the medical tracking application and one or more messaging systems selected from electronic mail, SMS text message, social media messaging and proprietary EMR messaging systems.

8. The method of claim 1 further comprising: adjusting, using the external computer, operational parameters from one or more messaging systems selected from electronic mail, SMS text message, social media messaging and proprietary EMR messaging systems.

9. (canceled)

10. (canceled)

11. (canceled)

12. The method of claim 1, wherein the codified medical elements direct the medical computer to use a short-range communication method to release one or more securely held medications being held for the client.

13. The method of claim 1, wherein the codified medical elements indicate health appliances, video equipment and environmental sensors that can be added to the medical computer and timing information for when each of the health appliances, video equipment and environmental sensors should be activated and used.

14. The method of claim 1, wherein the codified medical elements limit client activities, vital readings and environmental factors to raise alerts on the medical computer and to configured users at the external computer.

15. The method of claim 1, wherein the plurality of received medical activities triggers configured notification actions within the external computer, the configured notification actions comprising SMS messages, email messages, EMR signals and social media interactions.

16. A method of generating notifications or alerts based on medical activities relating to codified medical elements from a medical tracking application and managing client computer assignments to clients using an external computer, using one or more client assignment identifiers and the medical tracking application, the method comprising:
upon locating a medical tracking application on a medical computer, exchanging one or more client assignment identifier messages between the medical tracking application running on the medical computer and an external computer, wherein the one or more client assignment identifiers comprise or indicate capabilities of the medical computer allowing the external computer to determine whether the capabilities of the medical computer support requirements of the medical elements;
verifying the client using the one or more client assignment identifier messages;
upon verification of the client, receiving codified medical elements corresponding to the client from the external computer, wherein the codified medical elements comprise one or more required actions to fulfil the medical elements, the one or more required actions comprising at least one of biometric collection, barcode scanning, QR-code scanning, RFID communication and weight scale readings;
matching the capabilities of the medical computer against the requirements of the medical elements to further authenticate the capabilities of medical computer to support the medical tracking application to execute the codified medical elements;
executing the codified medical elements whereby the medical tracking application:
notifies the client when one or more medical elements have come due and at least one encoded action is required;
monitors the client performing the at least one required encoded action to indicate completion of the one or more medical elements;
determines whether each action Performed by the client was Performed correctly or incorrectly; and
reports on all inactions, actions and incorrect actions of the client related to the encoded medical elements.

17. The method of claim 16, wherein receiving codified medical elements comprises one or more actions to be performed by the client to confirm completion of necessary steps to fulfil the medical elements, including but not limited to barcode scanning, QR-code scanning, biometric collection, RFID communication, vital sign collection and weight scale readings.

18. The method of claim 16, wherein the incorrect actions comprise one or more failures to perform an action, the one or more failures to perform the action comprising at least one of performing the action in an incorrect way, performing the action early, performing the action late, performing the action excessively and not performing the action within a required time range.

19. The method of claim 16, wherein the one or more client assignment identifier messages to the external computer indicates that the medical computer used one or more stored client assignment identities to verify the client successfully.

20. A system for generating notifications or alerts based on medical activities relating to codified medical elements from a medical tracking application and assigning the medical tracking application to a client using an external computer, the system comprising:
at least one memory storing a client record with personal and medical elements and storing the client record in a database at an external computer, the client record corresponding to a client;
one or more databases at a medical computer and the external computer for storing one or more client assignment identifiers used for verifying an identity of a client;
a medical tracking application on the medical computer for exchanging one or more client assignment identifier messages allowing shared authentication of the client identity using one or more databases within the medical computer and the external computer; and
the external computer with at least one processor configured for:
exchanging one or more client assignment identifier messages to allow for the shared authentication of the client identity using the one or more databases within the external computer and the medical computer, wherein the one or more client assignment identifiers comprise or indicate capabilities of the medical computer allowing the external computer to determine whether the capabilities of the medical computer support requirements of the medical elements;
upon detecting that the medical tracking application is running on the medical computer, authenticating the client using the one or more client assignment identifier messages to verify the client identity and codify the medical elements from the client record for the client;
matching the capabilities of the medical computer against the requirements of the medical elements to further authenticate the capabilities of medical computer to support the medical tracking application to execute the codified medical elements, wherein the codified medical elements comprise one or more required actions to fulfil the medical elements, the one or more required actions comprising at least one of biometric collection, barcode scanning, QR-code scanning, RFID communication and weight scale readings;
sending the codified medical elements to the medical tracking application running on the medical computer for execution by the medical tracking application, the medical tracking application executing the codified medical elements to: notify the client when one or more medical elements have come due and at least one encoded action is required; monitor the client performing the at least one required encoded action using data received from the one or more connected medical devices; and determine whether each action Performed by the client was Performed correctly or incorrectly; and
receiving a plurality of medical activities related to the codified medical elements from the medical tracking application running on the medical computer;
generating a report and alerts for authorized users based on the plurality of medical activities relating to the codified medical elements, wherein the report comprises correct actions, incorrect actions, and inactions of the client related to the codified medical elements; and
wherein the external computer communicates with the medical tracking application running on the medical computer over at least one network.

21. The method of claim 1 further comprising processing the received plurality of medical activities using an artificial intelligence component to analyze patterns in the medical activities performed and not performed by the client, the artificial intelligence component making predictions regarding the client's future behaviour and adjusting one or more of the medical elements based on the predictions.

22. The method of claim 16 further comprising processing the received plurality of medical activities using an artificial intelligence component to analyze patterns in the medical activities performed and not performed by the client, the artificial intelligence component making predictions regarding the client's future behaviour and adjusting one or more of the medical elements based on the predictions.

23. The system of claim 20 further comprising an artificial intelligence component to analyze patterns in the medical activities performed and not performed by the client, the artificial intelligence component making predictions regarding the client's future behaviour and adjusting one or more of the medical elements based on the predictions.
